(19) United States
(12) Patent Application Publication
(10) Pub. No.: US 2005/0075493 A1
(43) Pub. Date: Apr. 7, 2005

(54) Flea synaptic vesicle nucleic acid molecules, proteins and uses thereof

(76) Inventor: Patrick J Gaines, Fort Collins, CO (US)

Correspondence Address: HESKA CORPORATION;INTELLECTUAL PROPERTY DEPT., 1613 PROSPECT PARKWAY, FORT COLLINS, CO, 80525, US

(21) Appl. No.: 10/399,215

(22) PCT Filed: Oct. 11, 2001
(86) PCT No.: PCT/US01/32122

Related U.S. Application Data
(63) Continuation-in-part of application No. 09/686,662, filed on Oct. 11, 2000, now abandoned.
Application No. 09/686,662 is a continuation-in-part of application No. 09/543,668, filed on Apr. 7, 2000, now abandoned.
(60) Provisional application No. 60/128,704, filed on Apr. 9, 1999.

(51) Int. Cl. C07H 21/04

(52) U.S. Cl. 536/23.5

(57) ABSTRACT

The present invention relates to flea synaptic vesicle proteins; to flea synaptic vesicle nucleic acid molecules, including those that encode such flea synaptic vesicle proteins, respectively; to antibodies raised against such proteins; and to compounds that inhibit the activity of such proteins. The present invention also includes methods to obtain such proteins, nucleic acid molecules, antibodies, and inhibitory compounds. The present invention also includes therapeutic compositions comprising such inhibitory compounds, particularly those that specifically inhibit flea synaptic vesicle activity, as well as the use of such therapeutic compositions to treat animals.

### FIELD OF THE INVENTION

The present invention relates to flea synaptic vesicle nucleic acid molecules, proteins encoded by such nucleic acid molecules, antibodies raised against such proteins, and inhibitors of such proteins. The present invention also includes methods to obtain such proteins, nucleic acid molecules, antibodies, and inhibitory compounds. The present invention also includes therapeutic compositions comprising such inhibitors, as well as uses thereof.

### BACKGROUND OF THE INVENTION

Flea infestation of animals is a health and economic concern for pet owners. In particular the bites of fleas are a problem for animals maintained as pets because the infestation becomes a source of annoyance not only for the pet but also for the pet owner who may find his or her home generally contaminated with insects. Fleas also directly cause a variety of diseases, including allergy, and also carry a variety of infectious agents including, but not limited to, endoparasites (e.g., nematodes, cestodes, trematodes and protozoa), bacteria and viruses. As such, fleas are a problem not only when they are on an animal but also when they are in the general environment of the animal.

The medical importance of flea infestation has prompted the development of reagents capable of controlling flea infestation. Commonly encountered methods to control flea infestation are generally focused on use of insecticides, which are often unsuccessful for one or more of the following reasons: (1) failure of owner compliance (frequent administration is required), (2) behavioral or physiological intolerance of the pet to the pesticide product or means of administration: and (3) the emergence of flea populations resistant to the prescribed dose of pesticide.

Synaptic vesicle proteins, including SVP1 an SVP2 proteins of the present invention, have structural and sequence conservation with a bacterial family of proton co-transporters, with the mammalian proton/glucose transporter, and with organic ion transporters. SVP has 12 putative transmembrane regions that arise from an internal duplication. In mammals, SVP proteins are located on neural and endocrine vesicles and are thought to function in the uptake of neurotransmitters into vesicles utilizing the proton gradient. Neurotransmitters in turn regulate the activity of the ion channels on these membranes. In the Malpighian tubules, the activity of tile ion channels determines the rate of diuresis, or fluid secretion from the hemolymph into tile lumen. Thus, inhibiting the transport of neurotransmitters in tile HMT tissues may have significant effects on tile functions of these tissues. As such, flea SVP1 and a SVP2 proteins of the present invention represent novel targets for anti-flea vaccines and chemotherapeutic drugs.

Therefore, isolation and sequencing of flea SVP1 and SVP2 genes may be critical for use in identifying specific agents for treating animals for flea infestation.

### SUMMARY OF THE INVENTION

The present invention provides flea synaptic vesicle nucleic acid molecules; proteins encoded by such nucleic acid molecules; antibodies raised against such proteins (i.e., anti-flea synaptic vesicle antibodies); mimetopes of such proteins or antibodies; and compounds that inhibit flea synaptic vesicle activity (i.e. inhibitory compounds or inhibitors).

The present invention also includes methods to obtain such proteins, mimetopes, nucleic acid molecules, antibodies and inhibitory compounds. The present invention also includes the use of proteins and antibodies to identify such inhibitory compounds as well as assay kits to identify such inhibitory compounds. Also included in the present invention are therapeutic compositions comprising proteins, mimetopes, nucleic acid molecules, antibodies and inhibitory compounds of the present invention including therapeutic compounds derived from a protein of the present invention that inhibit the activity of flea synaptic vesicle proteins; also included are uses of such therapeutic compounds.

One embodiment of the present invention is an isolated flea synaptic vesicle nucleic acid molecule that hybridizes with a nucleic acid sequence having SEQ ID NO:1, SEQ ID NO:2, SEQ ID NO:4, SEQ ID NO:5, SEQ ID NO:6, SEQ ID NO:7, SEQ ID NO:8. SEQ ID NO:10, SEQ ID NO:11, and/or SEQ ID NO:12, under conditions that allow less than or equal to 30% base pair mismatch. Another embodiment of tile present invention is an isolated flea synaptic vesicle nucleic acid molecule having a nucleic acid sequence that is at least 70% identical to SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and/or SEQ ID NO:12 and fragments of such proteins at least 10 amino acid residues in length.

The present invention also relates to recombinant molecules, recombinant viruses and recombinant cells that include a nucleic acid molecule of the present invention. Also included are methods to produce such nucleic acid molecules, recombinant molecules, recombinant viruses and recombinant cells.

Another embodiment of the present invention includes an isolated flea synaptic vesicle protein that is at least 70% identical to an amino acid sequence selected from the group consisting of SEQ ID NO:3 and/or SEQ ID NO:9 and fragments thereof at least 10 amino acid residues in length, wherein such fragments can elicit an immune response against respective flea synaptic vesicle proteins or have activity comparable to respective flea synaptic vesicle proteins.

Another embodiment of the present invention includes an isolated flea synaptic vesicle protein encoded by a nucleic acid molecule that hybridizes with a nucleic acid sequence having SEQ ID NO:4, SEQ ID NO:6, SEQ ID NO:10, and/or SEQ ID NO:12, under conditions that allow less than or equal to 30% base pair mismatch.

Another embodiment of the present invention includes a composition comprising an excipient and a compound selected from the group consisting of nucleic acid molecules, proteins, and antibodies of the present invention and a method to treat an animal for flea infestation comprising administering such a composition to such an animal.

Another embodiment of the present invention includes a method to detect an inhibitor of flea synaptic vesicle activity, said method comprising (a) contacting an isolated flea synaptic vesicle protein of the present invention, with a putative inhibitory compound under conditions in which, in the absence of said compound, said protein has flea synaptic vesicle protein activity, and (b) determining if said putative inhibitory compound inhibits flea synaptic vesicle protein activity.

### DETAILED DESCRIPTION OF THE INVENTION

The present invention provides for flea synaptic vesicle nucleic acid molecules, proteins encoded by such nucleic acid molecules, antibodies raised against such proteins, and inhibitors of such proteins. As used herein, flea synaptic vesicle nucleic acid molecules and proteins encoded by such nucleic acid molecules are also referred to as synaptic vesicle nucleic acid molecules and proteins, or SVP nucleic acid molecules and SVP proteins, respectively. Flea synaptic vesicle nucleic acid molecules and proteins of the present invention can be isolated from a flea or prepared recombinantly or synthetically. Flea synaptic vesicle nucleic acid molecules of the present invention can be RNA or DNA, or modified forms thereof, and can be double-stranded or single-stranded; examples of nucleic acid molecules include, but are not limited to, complementary DNA (cDNA) molecules, genomic DNA molecules, synthetic DNA molecules, DNA molecules which are specific tags for messenger RNA, and corresponding mRNA molecules. As such, a flea nucleic acid molecule of the present invention is not intended refer to an entire chromosome within which such a nucleic acid molecule is contained, however, a flea SVP cDNA of the present invention may include all regions such as regulatory regions that control production of flea SVP proteins encoded by such a cDNA (such as, but not limited to, transcription, translation or post-translation control regions) as well as the coding region itself, and any introns or non-translated coding regions. As used herein, the phrase “flea synaptic vesicle protein” refers to a protein encoded by a flea synaptic vesicle nucleic acid molecule.

Flea synaptic vesicle nucleic acid molecules of known length isolated from a flea, such as Ctenocephalides felis are denoted “nCfSVP1₈”, for example nCfSVP1₁₈₇₅, wherein “#” refers to the number of nucleotides in that molecule, and flea synaptic vesicle proteins of known length are denoted “PCfSVP1₈” (for example PCfSVP1₅₃₀) wherein “#” refers to the number of amino acid residues in that molecule.

The present invention also provides for flea synaptic vesicle DNA molecules that are specific tags for messenger RNA molecules. Such DNA molecules call correspond to an entire or partial sequence of a messenger RNA, and therefore, a DNA molecule corresponding to such a messenger RNA molecule (i.e. a cDNA molecule), can encode a full-length or partial-length protein. A nucleic acid molecule encoding a partial-length protein can be used directly as a probe or indirectly to generate primers to identify and/or isolate a cDNA nucleic acid molecule encoding a corresponding, or structurally related, full-length protein. Such a partial cDNA nucleic acid molecule can also be used in a similar manner to identify a genomic nucleic acid molecule, such as a nucleic acid molecule that contains the complete gene including regulatory regions, exons and introns. Methods for using partial flea synaptic vesicle cDNA molecules and sequences to isolate full-length and corresponding cDNA molecules are described in the examples herein below.

The proteins and nucleic acid molecules of the present invention can be obtained from their natural source, or can be produced using, for example, recombinant nucleic acid technology or chemical synthesis. Also included in the present invention is the use of these proteins and nucleic acid molecules as well as antibodies and inhibitory compounds thereto as therapeutic compositions to protect animals from flea infestation, as well as in other applications, such as those disclosed below.

One embodiment of the present invention is an isolated protein that includes a flea synaptic vesicle protein. It is to be noted that the term “a” or “an” entity refers to one or more of that entity; for example, a protein, a nucleic acid molecule, an antibody and a therapeutic composition refers to “one or more” or “at least one” protein, nucleic acid molecule, antibody and therapeutic composition respectively. As such, the terms “a” (or “an”), “one or more” and “at least one” can be used interchangeably herein. It is also to be noted that the terms “comprising”, “including”, and “having” can be used interchangeably. According to the present invention, an isolated, or biologically pure, protein, is a protein that has been removed from its natural milieu. As such, “isolated” and “biologically pure” do not necessarily reflect the extent to which the protein has been purified. An isolated protein of the present invention can be obtained from its natural source, can be produced using recombinant DNA technology, or can be produced by chemical synthesis.

As used herein, isolated flea synaptic vesicle proteins of the present invention can be full-length proteins or any homologue of such proteins. An isolated protein of the present invention, including a homologue, can be identified in a straight-forward manner by tile protein's ability to elicit an immune response against a flea synaptic vesicle protein or by the protein's ability to exhibit flea synaptic vesicle activity. Examples of flea synaptic vesicle homologue proteins include flea synaptic vesicle proteins in which amino acids have been deleted (e.g. a truncated version of the protein, such as a peptide), inserted, inverted, substituted and/or derivatized (e.g., by glycosylation, phosphorylation, acetylation, myristoylation, prenylation, palmitoylation, amidation and/or addition of glycerophosphatidyl inositol) such that the homologue includes at least one epitope capable of eliciting an immune response against a flea synaptic vesicle protein, and/or of binding to an antibody directed against a flea synaptic vesicle protein. That is, when the homologue is administered to an animal as an immunogen, using techniques known to those skilled in the art, the animal will produce an immune response against at least one epitope of a natural flea synaptic vesicle protein. The ability of a protein to effect an immune response call be measured using techniques known to those skilled in the art. As used herein, tile term “epitope” refers to the smallest portion of a protein or other antigen capable of selectively binding to the antigen binding site of an antibody or a T cell receptor. It is well accepted by those skilled in the art that the minimal size of a protein epitope is about four to six amino acids. As is appreciated by those skilled in the art, an epitope can include amino acids that naturally are contiguous to each other as well as amino acids that, due to the tertiary structure of the natural protein, ale in sufficiently close proximity to form an epitope. According to the present invention, an epitope includes a portion of a protein comprising at least 4 amino acids, at least 5 amino acids, at least 6 amino acids, at least 10 amino acids, at least 15 amino acids, at least 20 amino acids, at least 25 amino acids, at least 30 amino acids, at least 35 amino acids, at least 40 amino acids or at least 50 amino acids in length.

In one embodiment of the present invention a flea synaptic vesicle homologue protein has flea synaptic vesicle activity, i.e. the homologue exhibits all activity similar to its natural counterpart. Methods to detect and measure such activities ale known to those skilled in the art.

Flea synaptic vesicle homologue proteins can be the result of natural allelic variation or natural mutation. Flea synaptic vesicle protein homologues of the present invention can also be produced using techniques known in the art including, but not limited to, direct modifications to the protein or modifications to the gene encoding the protein using, for example, classic or recombinant DNA techniques to effect random or targeted mutagenesis.

Flea synaptic vesicle proteins of the present invention are encoded by flea synaptic vesicle nucleic acid molecules. As used herein, flea synaptic vesicle nucleic acid molecules include nucleic acid sequences related to natural flea synaptic vesicle genes, and, preferably, to C. felis flea synaptic vesicle genes. As used herein, flea synaptic vesicle genes include all regions such as regulatory regions that control production of flea synaptic vesicle proteins encoded by such genes (such as, but not limited to, transcription, translation or post-translation control regions) as well as the coding region itself, and any introns or non-translated coding regions. As used herein, a nucleic acid molecule that “includes” or “comprises” a sequence may include that sequence in one contiguous array, or may include the sequence as fragmented exons such as is often found for a flea gene. As used herein, the term “coding region” refers to a continuous linear array of nucleotides that translates into a protein. A full-length coding region is that coding region that is translated into a full-length, i.e., a complete protein as would be initially translated in its natural millieu, prior to any post-translational modifications.

One embodiment of the present invention is a C. felis flea synaptic vesicle gene that includes the nucleic acid sequence SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and/or SEQ ID NO:12. These nucleic acid sequences are further described herein. For example, nucleic acid sequence SEQ ID NO:2 represents the deduced sequence of the coding strand of a C. felis cDNA denoted herein as C. felis synaptic vesicle nucleic acid molecule nCfSVP1₁₈₇₅, the production of which is disclosed in the Examples. Nucleic acid molecule SEQ ID NO:2 comprises an apparently full-length coding region. The complement of SEQ ID NO:2 (represented herein by SEQ ID NO:4) refers to the nucleic acid sequence of the strand fully complementary to the strand halving SEQ ID NO:2, which can easily be determined by those skilled in the art. Likewise, a nucleic acid sequence complement of any nucleic acid sequence of the present invention refers to the nucleic acid sequence of the nucleic acid strand that is fully complementary to (i.e. can form a complete double helix with) the strand for which the sequence is cited. It should be noted that since nucleic acid sequencing technology is not entirely error-free. SEQ ID NO:2 (as well as other nucleic acid and protein sequences presented herein) represents an apparent nucleic acid sequence of the nucleic acid molecule encoding a flea synaptic vesicle protein of the present invention.

Translation of SEQ ID NO:2, the coding strand of nCfSVP1₁₈₇₅, as well as translation of SEQ ID NO:5, the coding strand of nCfSVP1₁₅₉₀, which represents the coding region of nCfSVP1₁₈₇₅, yields a protein of 530 amino acids, denoted herein as PCfSVP1₅₃₀, the amino acid sequence of which is presented in SEQ ID NO:3, assuming (a) an initiation codon extending from nucleotide 44 to 46 of SEQ ID NO:2, or from nucleotide 1 to nucleotide 3 of SEQ ID NO:5, respectively; and (b) a stop codon spanning from nucleotide 1634 to 1636 of SEQ ID NO:2.

Translation of SEQ ID NO:8, the coding strand of nCfSVP2₃₃₁₄, as well as translation of SEQ ID NO:11, the coding strand of nCfSVP2₂₃₁₉, which represents the coding region of nCfSVP2₃₃₁₄, yields a protein of 773 amino acids, denoted herein as PCfSVP2₇₇₃, the amino acid sequence of which is presented in SEQ ID NO:9, assuming (a) an initiation codon extending from nucleotide 302-304 of SEQ ID NO:8, or from nucleotide 1 to nucleotide 3 of SEQ ID NO:11, respectively; and (b) a stop codon spanning from nucleotide 2621 to 2623 of SEQ ID NO:8.

In one embodiment, a gene or other nucleic acid molecule of the present invention can be an allelic variant that includes a similar but not identical sequence to SEQ ID NO:1, SEQ ID NO:2, SEQ ID NO:4, SEQ ID NO:5, SEQ ID NO:6, SEQ ID NO:7, SEQ ID NO:8, SEQ ID NO:10. SEQ ID NO:11, and/or SEQ ID NO:12. For example, an allelic variant of a C. felis synaptic vesicle gene including SEQ ID NO:1, SEQ ID NO:2, SEQ LD NO:4, SEQ ID NO:5, SEQ ID NO:6, SEQ ID NO:7, SEQ ID NO:8, SEQ ID NO:10, SEQ ID NO:11, and/or SEQ ID NO:12 is a gene that occurs at essentially the same locus (or loci) in the genome as the gene including SEQ ID NO:1, SEQ ID NO:2. SEQ ID NO:4. SEQ ID NO:5. SEQ ID NO:6. SEQ ID NO:7, SEQ ID NO:8, SEQ ID NO:10, SEQ ID NO:11, and/or SEQ ID NO:12, but which, due to natural variations caused by, for example, mutation or recombination, has a similar but not identical sequence. Because natural selection typically selects against alterations that affect function, allelic variants (i.e. alleles corresponding to, or of, cited nucleic acid sequences) usually encode proteins having similar activity to that of the protein encoded by the gene to which they are being compared. Allelic variants of genes or nucleic acid molecules can also comprise alterations in tile 5′ or 3′ untranslated regions of the gene (e.g., in regulatory control regions), or can involve alternative splicing of a nascent transcript, thereby bringing alternative exons into juxtaposition. Allelic variants are well known to those skilled in the art and would be expected to occur naturally within a given flea species, since the genome is diploid, and sexual reproduction will result in the reassortment of alleles.

In one embodiment of the present invention, isolated flea synaptic vesicle proteins are encoded by nucleic acid molecules that hybridize under stringent hybridization conditions to genes or other nucleic acid molecules encoding flea synaptic vesicle proteins, respectively. The minimal size of flea synaptic vesicle proteins of the present invention is a size sufficient to be encoded by a nucleic acid molecule capable of forming a stable hybrid (i.e., hybridizing under stringent hybridization conditions) with the complementary sequence of a nucleic acid molecule encoding the corresponding natural protein. The size of a nucleic acid molecule encoding such a protein is dependent on the nucleic acid composition and the percent homology between the flea synaptic vesicle nucleic acid molecule and the complementary nucleic acid sequence. I can easily be understood that the extent of homology required to form a stable hybrid under stringent conditions can vary depending on whether the homologous sequences are interspersed throughout a given nucleic acid molecule or are clustered (i.e., localized) in distinct regions on a given nucleic acid molecule.

The minimal size of a nucleic acid molecule capable of forming a stable hybrid with a gene encoding a flea synaptic vesicle protein is at least about 12 to about 15 nucleotides in length if the nucleic acid molecule is CC-rich and at least about 15 to about 17 bases in length if it is AT-rich. The minimal size of a nucleic acid molecule used to encode a flea synaptic vesicle protein homologue of the present invention is from about 12 to about 18 nucleotides in length. Thus the minimal size of flea synaptic vesicle protein homologues of the present invention is from about 4 to about 6 amino acids in length. There is no limit, other than a practical limit, on the maximal size of a nucleic acid molecule encoding a flea synaptic vesicle protein of the present invention because a nucleic acid molecule of the present invention can include a portion of a gene or cDNA or RNA, an entire gene or cDNA or RNA, or multiple genes or cDNA or RNA. The preferred size of a protein encoded by a nucleic acid molecule of the present invention depends on whether a full-length, fusion, multivalent, or functional portion of such a protein is desired.

Stringent hybridization conditions are determined based on defined physical properties of the flea synaptic vesicle nucleic acid molecule to which the nucleic acid molecule is being hybridized, and can be defined mathematically. Stringent hybridization conditions are those experimental parameters that allow an individual skilled in the art to identify significant similarities between heterologous nucleic acid molecules. These conditions are well known to those skilled in the art. See, for example, Sambrook, et al., 1989, Molecular Cloning: A Laboratory Manual, Cold Spring Harbor Labs Press, and Meinkoth, et al., 1984, Anal, Biochem. 138, 267-284. As explained in detail in the cited references, the determination of hybridization conditions involves the manipulation of a set of variables including the ionic strength (M, in moles/liter), the hybridization temperature (° C.), the concentration of nucleic acid helix destabilizing agents (such as formamide), the average length of the shortest hybrid duplex (n), and the percent G+C composition of the fragment to which an unknown nucleic acid molecule is being hybridized. For nucleic acid molecules of at least about 150 nucleotides, these variables are inserted into a standard mathematical formula to calculate the melting temperature, Tₘ, of a given nucleic acid molecule. As defined in the formula below, Tₘ is the temperature at which two complementary nucleic acid molecule strands will disassociate, assuming 100% complementarity between the two strands:
Tₘ=81.5° C.+16.6 log M+0.41 (% G+C)−500/n−0.61 (% formamide).
For nucleic acid molecules smaller than about 50 nucleotides, hybrid stability is defined by the dissociation temperature (T_{d}), which is defined as the temperature at which 50% of the duplexes dissociate. For these smaller molecules, the stability at a standard ionic strength is defined by the following equation:
T_{d}=4(G+C)+2(A+T).
A temperature 5° C. below T_{d} is used to detect hybridization between perfectly matched molecules.

Also well known to those skilled in the art is how base pair mismatch, i.e. differences between two nucleic acid molecules being compared, including non-complementarity of bases at a given location, and gaps due to insertion or deletion of one or more bases at a given location on either of the nucleic acid molecules being compared, will affect Tₘ or T_{d} for nucleic acid molecules of different sizes. For example, Tₘ decreases about 1° C. for each 1% of mismatched base pairs for hybrids greater than about 150 bp, and T_{d} decreases about 5° C. For each mismatched base pair for hybrids below about 50 bp. Conditions for hybrids between about 50 and about 150 base pairs can be determined empirically and without undue experimentation using standard laboratory procedures well known to those skilled in the art. These simple procedures allow one skilled in the art to set the hybridization conditions (by altering, for example, the salt concentration, the concentration of helix destabilizing agents, or the temperature) so that only nucleic acid hybrids with greater than a specified % base pair mismatch will hybridize. Because one skilled in the art can easily determine whether a given nucleic acid molecule to be tested is less than or greater than about 50 nucleotides, and can therefore choose the appropriate formula for determining hybridization conditions, he or she can determine whether the nucleic acid molecule will hybridize with a given gene under conditions designed to allow a desired amount of base pair mismatch.

Hybridization reactions are often carried out by attaching the nucleic acid molecule to be hybridized to a solid support such as a membrane, and then hybridizing with a labeled nucleic acid molecule, typically referred to as a probe, suspended in a hybridization solution. Examples of common hybridization reaction techniques include, but are not limited to, the well-known Southern and northern blotting procedures. Typically, the actual hybridization reaction is done under non-stringent conditions. i.e. at a lower temperature and/or a higher salt concentration, and then high stringency is achieved by washing the membrane ill a solution with a higher temperature and/or lower salt concentration in order to achieve the desired stringency.

For example, if the skilled artisan wished to identify a nucleic acid molecule that hybridizes under conditions that would allow less thin or equal to 30% pail mismatch with a flea synaptic vesicle nucleic acid molecule of about 150 bp in length or greater, tile following conditions could preferably be used. Tile average G+C content of flea DNA is about 37%, as calculated from known flea nucleic acid sequences. The unknown nucleic acid molecules would be attached to a support membrane, and the 150 bp probe would be labeled, e.g. with a radioactive tag. The hybridization reaction could be carried out in a solution comprising 2×SSC in the absence of nucleic acid helix destabilizing compounds, at a temperature of about 37° C. (low stringency conditions). Solutions of differing concentrations of SSC can be made by one of skill in the art by diluting a stock solution of 20×SSC (175.3 gram NaCl and about 88.2 gram sodium citrate in 1 liter of water, pH 7) to obtain the desired concentration of SSC. The skilled artisan would calculate the washing conditions required to allow up to 30% base pair mismatch. For example, in a wash solution comprising 1×SSC in the absence of nucleic acid helix destabilizing compounds, the Tₘ of perfect hybrids would be about 77° C.:
81.5° C.+16.6 log (0.15M)+(0.41×73)−(500/150)−(0.61×0)=77.5° C.
Thus, to achieve hybridization with nucleic acid molecules having about 30% base pair mismatch, hybridization washes would be carried out at a temperature of less than or equal to 47.5° C. It is thus within tile skill of one in the art to calculate additional hybridization temperatures based on the desired percentage base pair mismatch, formulae and G/C content disclosed herein. For example, it is appreciated by one skilled in the art that as the nucleic acid molecule to be tested for hybridization against nucleic acid molecules of the present invention having sequences specified herein becomes longer than 150 nucleotides, the Tₘ for a hybridization reaction allowing up to 30% base pair mismatch will not vary significantly from 47.5° C.

Furthermore, it is known in the art that there ale commercially available computer programs for determining the degree of similarity between two nucleic acid or protein sequences. These computer programs include various known methods to determine the percentage identity and the number and length of gaps between hybrid nucleic acid molecules or proteins. Preferred methods to determine the percent identity among amino acid sequences and also among nucleic acid sequences include analysis using one or more of the commercially available computer programs designed to compare and analyze nucleic acid or amino acid sequences. These computer programs include, but are not limited to, the SeqLab® Wisconsin Package™ Version 10.0-UNIX sequence analysis software, available from Genetics Computer Group, Madison, Wis. (hereinafter “SeqLab”), and DNAsis® sequence analysis software, version 2.0, available from Hitachi Software, San Bruno, Calif. (hereinafter “DNAsis”). Such software programs represent a collection of algorithms paired with a graphical user interface for using the algorithms. The DNAsis and SeqLab software, for example, employ a particular algorithm, the Needleman-Wunsch algorithm to perform pair-wise comparisons between two sequences to yield a percentage identity score, see Needleman, S. B. and Wunch, C. D., 1970, J. Mol. Biol., 48, 443. Such algorithms, including the Needleman-Wunsch algorithm, are commonly used by those skilled in the nucleic acid and amino acid sequencing art to compare sequences. A preferred method to determine percent identity among amino acid sequences and also among nucleic acid sequences includes using the Needleman-Wunsch algorithm, available in the SeqLab software, using the Pairwise Comparison/Gap function with the nwsgapdna.cmp scoring matrix, the gap creation penalty and the gap extension penalties set at default values, and the gap shift limits set at maximum (hereinafter referred to as “SeqLab default parameters”). An additional preferred method to determine percent identity among amino acid sequences and also among nucleic acid sequences includes using the Higgins-Sharp algorithm, available in the DNAsis software, with the gap penalty set at 5, the number of top diagonals set at 5, the fixed gap penalty set at 10, the k-tuple set at 2, the window size set at 5, and the floating gap penalty set at 10. A particularly preferred method to determine percent identity among amino acid sequences and also among nucleic acid sequences includes using the Needleman-Wunsch algorithm available in the SeqLab software, using the SeqLab default parameters.

One embodiment of the present invention includes a flea synaptic vesicle protein. A preferred flea synaptic vesicle protein includes a protein encoded by a nucleic acid molecule that hybridizes under conditions that preferably allow less thin or equal to 30% base pair mismatch, preferably under conditions that allow less than or equal to 20% base pair mismatch, preferably under conditions that allow less than or equal to 10% base pair mismatch, preferably under conditions that allow less than or equal to 8% base pair mismatch, preferably under conditions that allow less than or equal to 5% base pair mismatch or preferably under conditions that allow less than or equal to 2% base pair mismatch with a nucleic acid molecule selected from the group consisting of SEQ ID NO:4, SEQ ID NO:6, SEQ ID NO:10, and/or SEQ ID NO:12.

Another embodiment of the present invention includes a flea synaptic vesicle protein encoded by a nucleic acid molecule that hybridizes under conditions comprising, (a) hybridizing in a solution comprising 1×SSC in the absence of nucleic acid helix destabilizing compounds, at a temperature of 37° C. and (b) washing in a solution comprising 1×SSC in the absence of nucleic acid helix destabilizing compounds, at a temperature of 47° C., to an isolated nucleic acid molecule selected from the group consisting of SEQ ID NO:4, SEQ ID NO:6, SEQ ID NO:10, and/or SEQ ID NO:12.

Another preferred flea synaptic vesicle protein of the present invention includes a protein that is encoded by a nucleic acid molecule that is preferably at least 70% identical, preferably at least 80% identical, preferably at least 90% identical, preferably at least 92% identical, preferably at least 95% identical or preferably at least 98% identical to a nucleic acid molecule having the nucleic acid sequence SEQ ID NO:1, SEQ ID NO:2, SEQ ID NO:5, SEQ ID NO:7, SEQ ID NO:8, and/or SEQ ID NO:11; also preferred are fragments (i.e. portions) of such proteins encoded by nucleic acid molecules that are at least 30nucleotides. Percent identity as used herein is determined using the Needleman-Wunsch algorithm, available in the SeqLab software using default parameters.

Additional preferred flea synaptic vesicle proteins of the present invention include proteins having the amino acid sequence SEQ ID NO:3 and/or SEQ ID NO:9, and proteins comprising homologues of a protein having the albino acid sequence SEQ ID NO:3 aid/or SEQ ID NO:9, wherein such a homologue comprises at least one epitope that elicits an immune response against a protein having an amino acid sequence SEQ ID NO:3 and/or SEQ ID NO:9. Likewise, also preferred are proteins encoded by nucleic acid molecules comprising nucleic acid sequence SEQ ID NO:1, SEQ ID NO:2, SEQ ID NO:5. SEQ ID NO:7, SEQ ID NO:8, and/or SEQ ID NO:11, or by homologues thereof.

A preferred isolated flea synaptic vesicle protein of the present invention is a protein encoded by at least one of the following nucleic acid molecules: nCfSVP1₁₈₇₅, nCfSVP1₁₅₉₀, nCfSVP2₃₃₁₄, and/or nCfSVP2₂₃₁₉, or allelic variants of any of these nucleic acid molecules. Also preferred is an isolated protein encoded by a nucleic acid molecule having nucleic acid sequence SEQ ID NO:1, SEQ ID NO:2, SEQ ID NO:5, SEQ ID NO:7, SEQ ID NO:8, and/or SEQ ID NO:11; or a protein encoded by an allelic variant of any of these listed nucleic acid molecules.

Preferred flea synaptic vesicle proteins of the present invention include proteins having amino acid sequences that are at least 70%, preferably 80%, preferably 90%, preferably 92%, preferably 95%, preferably at least 98%, preferably at least 99%, or preferably 100% identical to amino acid sequence SEQ ID NO:3 and/or SEQ ID NO:9 and proteins encoded by allelic variants of nucleic acid molecules encoding flea synaptic vesicle proteins having amino acid sequences SEQ ID NO:3 and/or SEQ ID NO:9. Also preferred are fragments thereof having at least 10 amino acid residues.

In one embodiment of the present invention, C. felis synaptic vesicle proteins comprise amino acid sequence SEQ ID NO:3 and/or SEQ ID NO:9 (including, but not limited to, the proteins consisting of amino acid sequence SEQ ID NO:3 and/or SEQ ID NO:9, fusion proteins and multivalent proteins), and proteins encoded by allelic variants of nucleic acid molecules encoding proteins having amino acid sequence SEQ ID NO:3 and/or SEQ ID NO:9.

In one embodiment, a preferred flea synaptic vesicle protein comprises an amino acid sequence of at least 6 amino acids, preferably at least 10 amino acids, preferably at least 15 amino acids, preferably at least 20 amino acids, preferably at least 25 amino acids, preferably at least 30 amino acids, preferably at least 35 amino acids, preferably at least 40 amino acids, preferably at least 50 amino acids, preferably at least 75 amino acids, preferably at least 100 amino acids, preferably at least 125 amino acids, preferably at least 150 amino acids, preferably at least 175 amino acids, preferably at least 200 amino acids, preferably at least 250 amino acids, preferably at least 300 amino acids, preferably at least 350 amino acids, preferably at least 400 amino acids, preferably at least 450 amino acids, preferably at least 500 amino acids, preferably at least 550 amino acids, preferably at least 600 amino acids, preferably at least 650 amino acids, preferably at least 700 amino acids, preferably at least 750 amino acids, or preferably at least 775 amino acids. In another embodiment, preferred flea synaptic vesicle proteins comprise full-length proteins, i.e., proteins encoded by full-length coding regions, or post-translationally modified proteins thereof, such as mature proteins from which initiating methionine and/or signal sequences or “pro” sequences have been removed.

Additional preferred flea synaptic vesicle proteins of the present invention include proteins encoded by nucleic acid molecules comprising at least a portion of nCfSVP1₁₈₇₅, nCfSVP1₁₅₉₀, nCfSVP2₃₃₁₄, and/or nCfSVP2₂₃₁₉, as well as flea synaptic vesicle proteins encoded by allelic variants of such nucleic acid molecules. A portion of such flea synaptic vesicle nucleic acid molecule is preferably at least 30 nucleotides in length.

Also preferred are flea synaptic vesicle proteins encoded by nucleic acid molecules having nucleic acid sequences comprising at least a portion of SEQ ID NO:1, SEQ ID NO:2, SEQ ID NO:5, SEQ ID NO:7, SEQ ID NO:8, and/or SEQ ID NO:11, as well as allelic variants of these nucleic acid molecules. A portion of such flea synaptic vesicle nucleic acid molecule is preferably at least 30nucleotides in length.

In another embodiment, a preferred flea synaptic vesicle protein of the present invention is encoded by a nucleic acid molecule comprising at least 20 nucleotides, preferably at least 25 nucleotides, preferably at least 30 nucleotides, preferably at least 40 nucleotides, preferably at least 50 nucleotides, preferably at least 75 nucleotides, preferably at least 100 nucleotides, preferably at least 200 nucleotides, preferably at least 400 nucleotides, preferably at least 500 nucleotides, preferably at least 750 nucleotides, preferably at least 1000 nucleotides, preferably at least 1500 nucleotides, preferably at least 1800 nucleotides, preferably at least 2000 nucleotides, preferably at least 2300 nucleotides, or preferably at least 3300 nucleotides in length. Within this embodiment is a flea synaptic vesicle protein encoded by at least a portion of nCfSVP1₁₈₇₅, nCfSVP1₁₅₉₀, nCfSVP2₃₃₁₄, and/or nCfSVP2₂₃₁₉, or by an allelic variant of any of these nucleic acid molecules. Preferred flea synaptic vesicle proteins of the present invention are encoded by nucleic acid molecules comprising apparently full-length flea synaptic vesicle coding region, i.e., nucleic acid molecules encoding an apparently full-length flea synaptic vesicle protein.

Preferred flea synaptic vesicle proteins of the present invention can be used to develop inhibitors that, when administered to an animal in an effective manner, are capable of protecting that animal from flea infestation. In accordance with the present invention, the ability of an inhibitor of the present invention to protect an animal from flea infestation refers to the ability of that protein to, for example, treat, ameliorate and/or prevent infestation caused by fleas. In particular, the phrase “to protect an animal from flea infestation” refers to reducing the potential for flea population expansion on and around the animal (i.e., reducing the flea burden). Preferably, the flea population size is decreased, optimally to an extent that the animal is no longer bothered by fleas. A host animal, as used herein, is an animal from which fleas can feed by attaching to and feeding through the skin of the animal. Fleas, and other ectoparasites, can live on a host animal for an extended period of time or can attach temporarily to an animal in order to feed. At any given time, a certain percentage of a flea population can be on a host animal whereas the remainder can be in the environment of the animal. Such an environment can include not only adult fleas, but also flea eggs and/or flea larvae. The environment can be of any size such that fleas in the environment are able to jump onto and off of a host animal. For example, the environment of an animal can include plants, such as crops, from which fleas infest an animal. As such, it is desirable not only to reduce the flea burden on an animal per se, but also to reduce the flea burden in the environment of the animal.

Suitable fleas to target include any flea that is essentially incapable of causing disease in ail animal administered an inhibitor of the present invention. As such, fleas to target include any flea that produces a protein that can be targeted by an inhibitory compound that inhibits a flea flea synaptic vesicle protein function, thereby resulting in the decreased ability of the parasite to cause disease in an animal. Preferred fleas to target include fleas of the following genera: Ctenocephalides, Cyopsyllus, Diamanus (Oropsylla), Echidnophaga, Nosopsyllus, Pulex, Tunga, and Xenopsylla, with those of the species Ctenocephalides canis, Ctenocephalides felis, Diamanus montanus, Echidnophaga gallinacea, Nosopsyllus faciatus, Pulex irritans, Pulex simulans, Tunga penetrans and Xenopsylla cheopis being more preferred, with C. felis being even more preferred. Such fleas are also preferred for the isolation of proteins or nucleic acid molecules of the present invention.

One embodiment of a flea synaptic vesicle protein of the present invention is a fusion protein that includes a flea synaptic vesicle protein-containing domain attached to one or more fusion segments. Suitable fusion segments for use with the present invention include, but are not limited to, segments that can: enhance a proteins stability; act as an immunopotentiator; and/or assist in purification of a flea synaptic vesicle protein (e.g., by affinity chromatography). A suitable fusion segment can be a domain of any size that has the desired function (e.g., imparts increased stability, imparts increased immunogenicity to a protein, and/or simplifies purification of a protein). Fusion segments can be joined to amino and/or carboxyl termini of the flea synaptic vesicle-containing domain of the protein and can be susceptible to cleavage in order to enable straight-forward recovery of a flea synaptic vesicle protein. Fusion proteins are preferably produced by culturing a recombinant cell transformed with a fusion nucleic acid molecule that encodes a protein including the fusion segment attached to either the carboxyl and/or amino terminal end of a flea synaptic vesicle-containing domain. Preferred fusion segments include a metal binding domain (e.g., a poly-histidine segment); an immunoglobulin binding domain (e.g., Protein A; Protein G; T cell; B cell: Fc receptor or complement protein antibody-binding domains): a sugar binding domain (e.g., a maltose binding domain); and/or a “tag” domain (e.g., at least a portion of β-galactosidase, a strep tag peptide, a T7 tag peptide, a Flag™ peptide, or other domains that can be purified using compounds that bind to the domain, such as monoclonal antibodies). More preferred fusion segments include metal binding domains, such as a poly-histidine segment; a maltose binding domain; a strep tag peptide, such as that available from Biometra in Tampa, Fla.; and an S10 peptide.

The present invention also includes mimetopes of flea synaptic vesicle proteins of the present invention. As used herein, a mimetope of a flea synaptic vesicle protein of the present invention refers to ally compound that is able to mimic the activity of such a flea synaptic vesicle protein, often because the mimetope has a structure that mimics the particular flea synaptic vesicle protein. Mimetopes can be, but are not limited to: peptides that have been modified to decrease the susceptibility to degradation such as all-D retro peptides; anti-idiotypic and/or catalytic antibodies, or fragments thereof; non-proteinaceous immunogenic portions of an isolated protein (e.g., carbohydrate structures); and synthetic or natural organic molecules, including nucleic acids. Such mimetopes can be designed using computer-generated structures of proteins of the present invention. Mimetopes can also be obtained by generating random samples of molecules, such as oligonucleotides, peptides or other organic molecules, and screening such samples by affinity chromatography techniques using the corresponding binding partner.

Another embodiment of the present invention is an isolated nucleic acid molecule comprising a flea synaptic vesicle nucleic acid molecule, i.e. a nucleic acid molecule that can be isolated from a flea cDNA library. As used herein, flea synaptic vesicle nucleic acid molecules has the same meaning as flea synaptic vesicle nucleic acid molecule. The identifying characteristics of such nucleic acid molecules are heretofore described. A nucleic acid molecule of the present invention can include an isolated natural flea synaptic vesicle gene or a homologue thereof, the latter of which is described in more detail below. A nucleic acid molecule of tile present invention can include one or more regulatory regions, full-length or partial coding regions, or combinations thereof. The minimal size of a nucleic acid molecule of the present invention is a size sufficient to allow the formation of a stable hybrid (i.e., hybridization under stringent hybridization conditions) with the complementary sequence of another nucleic acid molecule. As such, the minimal size of a flea synaptic vesicle nucleic acid molecule of the present invention is from about 12 to about 18 nucleotides in length.

In accordance with the present invention, an isolated nucleic acid molecule is a nucleic acid molecule that has been removed from its natural milieu (i.e., that has been subjected to human manipulation) and can include DNA, RNA, or derivatives of either-DNA or RNA. As such, “isolated” does not reflect the extent to which the nucleic acid molecule has been purified. Isolated flea synaptic vesicle nucleic acid molecules of the present invention, or homologues thereof, can be isolated from a natural source or produced using recombinant DNA technology (e.g., polymerase chain reaction (PCR) amplification or cloning) or chemical synthesis. Isolated flea synaptic vesicle nucleic acid molecules, and homologues thereof, can include, for example, natural allelic variants and nucleic acid molecules modified by nucleotide insertions, deletions, substitutions, and/or inversions in a manner such that the modifications do not substantially interfere with the nucleic acid molecule's ability to encode a flea synaptic vesicle protein of the present invention.

A flea synaptic vesicle nucleic acid molecule homologue can be produced using a number of methods known to those skilled in the art, see, for example, Sambrook et al., ibid. For example, nucleic acid molecules can be modified using a variety of techniques including, but not limited to, classic mutagenesis and recombinant DNA techniques such as site-directed mutagenesis, chemical treatment, restriction enzyme cleavage, ligation of nucleic acid fragments, PCR amplification, synthesis of oligonucleotide mixtures and ligation of mixture groups to “build” a mixture of nucleic acid molecules, and combinations thereof. Nucleic acid molecule homologues can be selected by hybridization with flea synaptic vesicle nucleic acid molecules or by screening the function of a protein encoded by the nucleic acid molecule (e.g., ability to elicit an immune response against at least one epitope of a flea synaptic vesicle protein or to effect flea synaptic vesicle activity).

An isolated flea synaptic vesicle nucleic acid molecule of the present invention call include a nucleic acid sequence that encodes at least one flea synaptic vesicle protein of the present invention respectively, examples of such proteins being disclosed herein. Although the phrase “nucleic acid molecule” primarily refers to tile physical nucleic acid molecule and the phrase “nucleic acid sequence” primarily refers to the sequence of nucleotides on the nucleic acid molecule, the two phrases call be used interchangeably, especially with respect to a nucleic acid molecule, or a nucleic acid sequence, being capable of encoding a flea synaptic vesicle protein.

A preferred nucleic acid molecule or the present invention, when administered to an animal, is capable of protecting that animal from flea infestation. As will be disclosed in more detail below, a nucleic acid molecule of the present invention can be, or encode, an antisense RNA, a molecule capable of triple helix formation, a ribozyme, or other nucleic acid-based drug compound. In additional embodiments, a nucleic acid molecule of the present invention can encode a protective protein (e.g., a flea synaptic vesicle protein of the present invention), the nucleic acid molecule being delivered to the animal, for example, by direct injection (i.e, as a genetic vaccine) or in a vehicle such as a recombinant virus vaccine or a recombinant cell vaccine.

In one embodiment of the present invention, a preferred flea synaptic vesicle nucleic acid molecule includes an isolated nucleic acid molecule that hybridizes under conditions that preferably allow less than or equal to 30% base pair mismatch, preferably under conditions that allow less than or equal to 20% base pair mismatch, preferably under conditions that allow less than or equal to 10% base pair mismatch preferably under conditions that allow less than or equal to 5% base pair mismatch or preferably under conditions that allow less than or equal to 2% base pair mismatch with a nucleic acid molecule selected from the group consisting of SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and/or SEQ ID NO:12.

Another embodiment of the present invention includes a flea synaptic vesicle nucleic acid molecule, wherein said nucleic acid molecule hybridizes under conditions comprising, (a) hybridizing in solution comprising 1×SSC in the absence of nucleic acid helix destabilizing compounds, at a temperature of 37° C. and (b) washing in a solution comprising 1×SSC in the absence of nucleic acid helix destabilizing compounds, at a temperature of 47° C., to an isolated nucleic acid molecule selected from the group consisting of S SEQ ID NO:1, SEQ ID NO:2, SEQ ID NO:4, SEQ ID NO:5. SEQ ID NO:6, SEQ ID NO:7, SEQ ID NO:8, SEQ ID NO:10, SEQ ID NO:11, and/or SEQ ID NO:12. Additional preferred nucleic acid molecules of the present invention include oligonucleotides of an isolated nucleic acid molecule, wherein said nucleic acid molecule hybridizes under conditions comprising, (a) hybridizing in solution comprising 1×SSC in the absence of nucleic acid helix destabilizing compounds, it a temperature of 37° C. and (b) washing in a solution comprising 1×SSC in the absence of nucleic acid helix destabilizing compounds, at a temperature of 47° C., to an isolated nucleic acid molecule selected from the group consisting of SEQ ID NO:1, SEQ ID NO:2, SEQ ID NO:4, SEQ ID NO:5, SEQ ID NO:6, SEQ ID NO:7. SEQ ID NO:8, SEQ ID NO:10, SEQ ID NO:11, and/or SEQ ID NO:12, wherein said oligonucleotide comprises at least 30nucleotides.

Additional preferred flea synaptic vesicle nucleic acid molecules of the present invention include nucleic acid molecules comprising a nucleic acid sequence that is preferably at least 70%, preferably at least 80%, preferably at least 90%, preferably at least 92%, preferably at least 95%, or preferably at least 98% identical to a nucleic acid sequence selected from the group consisting of SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and/or SEQ ID NO:12. Also preferred are oligonucleotides of any of such nucleic acid molecules. Percent identity as used herein is determined using the Needleman-Wunsch algorithm, available in the SeqLab software using default parameters.

One embodiment of the present invention is a nucleic acid molecule comprising all or part of nucleic acid molecules nCfSVP1₁₈₇₅, nCfSVP1₁₅₉₀, nCfSVP2₃₃₁₄, and/or nCfSVP2₂₃₁₉, or allelic variants of these nucleic acid molecules Another preferred nucleic acid molecule of the present invention includes at least a portion of nucleic acid sequence SEQ ID NO:1, SEQ ED NO:2, SEQ ID NO:4, SEQ ID NO:5, SEQ ID NO:6, SEQ ID NO:7, SEQ ID NO:8, SEQ ID NO:10, SEQ ID NO:11, and/or SEQ ID NO:12, as well as allelic variants of nucleic acid molecules having these nucleic acid sequences and homologues of nucleic acid molecules having these nucleic acid sequences: preferably such a homologues encodes or is complementary to a nucleic acid molecule that encodes at least one epitope that elicits an immune response against a protein having an amino acid sequence SEQ ID NO:3 and/or SEQ ID NO:9. Such nucleic acid molecules acid include nucleotides in addition to those included in the SEQ ID NOs, such as, but not limited to, a full-length gene, a full-length coding region, a nucleic acid molecule encoding a fusion protein, or a nucleic acid molecule encoding, a multivalent protective compound.

In one embodiment, a flea synaptic vesicle nucleic acid molecule of the present invention encodes a protein having an amino acid sequence that is at least 70%, preferably at least 80%, preferably at least 90%, preferably at least 95%, preferably at least 98%, preferably at least 99%, or preferably at least 100% identical to SEQ ID NO:3 and/or SEQ ID NO:9. The present invention also includes a flea synaptic vesicle nucleic acid molecule encoding a protein having at least a portion of SEQ ID NO:3 and/or SEQ ID NO:9, as well as allelic variants of a nucleic acid molecule encoding a protein having these sequences, including nucleic acid molecules that have been modified to accommodate codon usage properties of the cells in which such nucleic acid molecules are to be expressed.

In another embodiment, a preferred flea synaptic vesicle nucleic acid molecule of the present invention comprises a nucleic acid molecule comprising at least 20 nucleotides, preferably at least 25 nucleotides, preferably at least 30 nucleotides, preferably at least 40 nucleotides, preferably at least 50 nucleotides, preferably at least 75 nucleotides, preferably at least 100 nucleotides, preferably at least 200 nucleotides, preferably at least 400 nucleotides, preferably at least 500 nucleotides, preferably at least 750 nucleotides, preferably at least 1000 nucleotides, preferably at least 1500 nucleotides, preferably at least 1800 nucleotides, preferably at least 2000 nucleotides, preferably at least 2300 nucleotides, or preferably at least 3300 nucleotides in length.

In another embodiment, a preferred flea synaptic vesicle nucleic acid molecule encodes a protein comprising at least 6 amino acids, preferably at least 10 amino acids, preferably at least 20 amino acids, preferably at least 30 amino acids, preferably at least 40 amino acids, preferably at least 50 amino acids, preferably at least 75 amino acids, preferably at least 100 amino acids, preferably at least 125 amino acids, preferably at least 150 amino acids, preferably at least 175 amino acids, preferably at least 200 amino acids, preferably at least 250 amino acids, preferably at least 300 amino acids, preferably at least 350 amino acids, preferably at least 400 amino acids, preferably at least 450 amino acids, preferably at least 500 amino acids, preferably at least 550 amino acids, preferably at least 600 amino acids, preferably at least 650 amino acids, preferably at least 700 amino acids, preferably at least 750 amino acids, or preferably at least 775 amino acids.

In another embodiment, a preferred flea synaptic vesicle nucleic acid molecule of the present invention comprises in apparently full-length flea synaptic vesicle coding region, i.e., the preferred nucleic acid molecule encodes an apparently full-length flea synaptic vesicle protein, respectively, or a post-translationally modified protein thereof. In one embodiment, a preferred flea synaptic vesicle nucleic acid molecule of the present invention encodes a mature protein.

In another embodiment, a preferred flea synaptic vesicle nucleic acid molecule of the present invention comprises a nucleic acid molecule comprising SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and/or SEQ ID NO:12, or a fragment thereof.

A fragment of a flea synaptic vesicle nucleic acid molecule of the present invention preferably comprises at least 18 nucleotides, preferably at least 21 nucleotides, preferably at least 25 nucleotides, preferably at least 30 nucleotides, preferably at least 35 nucleotides, preferably at least 40 nucleotides, preferably at least 50 nucleotides, preferably at least 75 nucleotides, preferably at least 100 nucleotides, preferably at least 200 nucleotides, preferably at least 400 nucleotides, preferably at least 500 nucleotides, preferably at least 750 nucleotides, preferably at least 1000 nucleotides, preferably at least 1500 nucleotides, preferably at least 1800 nucleotides, preferably at least 2000 nucleotides, preferably at least 2300 nucleotides, or preferably at least 3300 nucleotides identical in sequence to a corresponding contiguous sequence selected from the group consisting of SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and/or SEQ ID NO:12.

The phrase, a nucleic acid molecule comprising at least “x” contiguous, or consecutive nucleotides identical in sequence to at least “x” contiguous, or consecutive nucleotides of a nucleic acid molecule selected from tile group consisting of SEQ ID NO:“y”, refers to an “x”-nucleotide in length nucleic acid molecule that is identical in sequence to an “x”-nucleotide portion of SEQ ID NO:“y”, as well as to nucleic acid molecules that are longer in length than “x”. The additional length may be in the form of nucleotides that extend from either, tile 5′ or the 3′ end(s) of the contiguous identical “x”-nucleotide portion. The 5′ and/or 3′ extensions can include one or more extensions that have no identity to a molecule of tile present invention, as well as extensions that show similarity or identity to cited nucleic acids sequences or portions thereof.

Knowing the nucleic acid sequences of certain flea synaptic vesicle nucleic acid molecules of the present invention allows one skilled in the art to, for example, (a) make copies of those nucleic acid molecules, (b) obtain nucleic acid molecules including at least a portion of such nucleic acid molecules (e.g., nucleic acid molecules including full-length genes, full-length coding regions, regulatory control sequences, truncated coding regions), and (c) obtain other flea synaptic vesicle nucleic acid molecules. Such nucleic acid molecules can be obtained in a variety of ways including screening appropriate expression libraries with antibodies of the present invention; traditional cloning techniques using oligonucleotide probes of the present invention to screen appropriate libraries; and PCR amplification of appropriate libraries or DNA using oligonucleotide primers of the present invention. Preferred libraries to screen or from which to amplify nucleic acid molecules include cDNA libraries as well as genomic DNA libraries. Similarly, preferred DNA sources to screen or from which to amplify nucleic acid molecules include cDNA and genomic DNA. Techniques to clone and amplify genes are disclosed, for example, in Sambrook et al., ibid.

The present invention also includes nucleic acid molecules that arc oligonucleotides capable of hybridizing, under stringent hybridization conditions, with complementary regions of other, preferably longer, nucleic acid molecules of the present invention such as those comprising C. felis synaptic vesicle nucleic acid molecules or other flea synaptic vesicle nucleic acid molecules. Oligonucleotides of the present invention can be RNA, DNA, or derivatives of either. The minimum size of such oligonucleotides is the size required for formation of a stable hybrid between an oligonucleotide and a complementary sequence on a nucleic acid molecule of tile present invention. A preferred oligonucleotide of the present invention has at maximum size of preferably 100 to 200 nucleotides. The present invention includes oligonucleotides that can be used as, for example, probes to identify nucleic acid molecules, primers to produce nucleic acid molecules, or therapeutic reagents to inhibit flea synaptic vesicle protein production or activity (e.g., as antisense-, triplex formation-, ribozyme- and/or RNA drug-based reagents). The present invention also includes tile use of such oligonucleotides to protect animals from disease using one or more of such technologies. Appropriate oligonucleotide-containing therapeutic compositions can be administered to an animal using techniques known to those skilled in the art.

One embodiment of the present invention includes a recombinant vector, which includes at least one isolated nucleic acid molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid. Recombinant vectors can be used in the cloning, sequencing, and/or otherwise manipulating of flea synaptic vesicle nucleic acid molecules of the present invention.

One type of recombinant vector, referred to herein as a recombinant molecule, comprises a nucleic acid molecule of the present invention operatively linked to an expression vector. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, parasite, insect, other animal, and plant cells. Preferred expression vectors of the present invention call direct gene expression in bacterial, yeast, insect and mammalian cells, and more preferably in the cell types disclosed herein.

In particular, expression vectors of the present invention contain regulatory sequences such as transcription control sequences, translation control sequences, origins of replication, and other regulatory sequences that are compatible with the recombinant cell and that control the expression of nucleic acid molecules of tile present invention. In particular, recombinant molecules of tile present invention include transcription control sequences. Transcription control sequences are sequences that control the initiation, elongation, and termination of transcription. Particularly important transcription control sequences ale those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those that function in bacterial, yeast, or insect and mammalian cells, such as, but not limited to, tac, lac, trp, trc, oxy-pro, omp/lpp, rrnB, bacteriophage lambda (such as lambda p_{L} and lambda p_{R} and fusions that include such promoters), bacteriophage T7, T7lac, bacteriophage T3, bacteriophage SP6, bacteriophage SP01, metallothionein, alpha-mating factor, Pichia alcohol oxidase, alphavirus subgenomic promoter, antibiotic resistance gene, baculovirus, Heliothis zea insect virus, vaccinia virus, herpesvirus, raccoon poxvirus, other poxvirus, adenovirus, cytomegalovirus (such as immediate early promoter), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences of the present invention can also include naturally occurring transcription control sequences naturally associated with fleas, such as C. felis transcription control sequences.

Suitable and preferred nucleic acid molecules to include in recombinant vectors of the present invention are as disclosed herein. Preferred nucleic acid molecules to include in recombinant vectors, and particularly in recombinant molecules, include nCfSVP1₁₈₇₅, nCfSVP1₁₅₉₀, nCfSVP2₃₃₁₄, and/or nCfSVP2₂₃₁₉.

Recombinant molecules of the present invention may also (a) contain secretory signals (i.e., signal segment nucleic acid sequences) to enable all expressed flea synaptic vesicle protein of the present invention to be secreted from the cell that produces the protein and/or (b) contain fusion sequences which lead to the expression of nucleic acid molecules of the present invention as fusion proteins. Examples of suitable signal segments include any signal segment capable of directing the secretion of a protein of the present invention. Preferred signal segments include, but are not limited to, tissue plasminogen activator (t-PA), interferon, interleukin, growth hormone, histocompatibility and viral envelope glycoprotein signal segments. Suitable fusion segments encoded by fusion segment nucleic acids are disclosed herein. In addition, a nucleic acid molecule of the present invention can be joined to a fusion segment that directs the encoded protein to the proteosome, such as a ubiquitin fusion segment. Eukaryotic recombinant molecules may also include intervening and/or untranslated sequences surrounding and/or within the nucleic acid sequences of nucleic acid molecules of the present invention.

Another embodiment of the present invention includes a recombinant cell comprising a host cell transformed with one or more recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. It is to be noted that a cell line refers to any recombinant cell of the present invention that is not a transgenic animal. Transformed nucleic acid molecules of the present invention can remain extrachromosomal or can integrate into one or more sites within a chromosome of the transformed (i.e., recombinant) cell in such a manner that their ability to be expressed is retained. Preferred nucleic acid molecules with which to transform a cell include flea synaptic vesicle nucleic acid molecules disclosed herein. Preferred nucleic acid molecules with which to transform a cell include nCfSVP1₁₈₇₅, nCfSVP1₁₅₉₀, nCfSVP2₃₃₁₄, and/or nCfSVP2₂₃₁₉.

Suitable host cells to transform include ally cell that can be transformed with it nucleic acid molecule of the present invention. Host cells can be either untransformed cells or cells that are already transformed with at least one nucleic acid molecule (e.g., nucleic acid molecules encoding one or more proteins of the present invention and/or other proteins useful in the production of multivalent vaccines). Host cells of the present invention either can be endogenously (i.e., naturally) capable of producing flea synaptic vesicle proteins of the present invention or can be capable of producing such proteins after being transformed with at least one nucleic acid molecule of the present invention. Host cells of the present invention can be any cell capable of producing at least one protein of the present invention, and include bacterial, fungal (including yeast), parasite (including helminth, protozoa and ectoparasite), other insect, other animal and plant cells. Preferred host cells include bacterial, mycobacterial, yeast, insect and mammalian cells. More preferred host cells include Salmonella, Escherichia, Bacillus, Caulobacter, Listeria, Saccharomyces, Pichia, Spodoptera, Mycobacteria, Trichoplusia, BHK (baby hamster kidney) cells, MDCK cells (Madin-Darby canine kidney cell line), CRFK cells (Crandell feline kidney cell line), CV-1 cells (African monkey kidney cell line used, for example, to culture raccoon poxvirus), COS (e.g., COS-7) cells, and Vero cells. Particularly preferred host cells are Escherichia coli, including E. coli K-12 derivatives; Salmonella typhi; Salmonella typhimurium, including attenuated strains such as UK-1 ₓ3987 and SR-11, ₓ4072; Caulobacter; Pichia; Spodoptera frugiperda; Trichoplusia ni; BHK cells; MDCK cells; CRFK cells; CV-1 cells; COS cells; Vero cells; and non-tumorigenic mouse myoblast G8 cells (e.g., ATCC CRL 1246). Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK³¹ cells and/or HeLa cells. In one embodiment, the proteins may be expressed as heterologous proteins in myeloma cell lines employing immunoglobulin promoters.

A recombinant cell is preferably produced by transforming a host cell with one or more recombinant molecules, each comprising one or more nucleic acid molecules of the present invention operatively linked to an expression vector containing one or more transcription control sequences, examples of which are disclosed herein. The phrase operatively linked refers to insertion of a nucleic acid molecule into an expression vector in a manner such that the molecule is able to be expressed when transformed into a host cell.

A recombinant cell of the present invention includes any cell transformed with at least one of any nucleic acid molecule of the present invention. Suitable and preferred nucleic acid molecules as well as suitable and preferred recombinant molecules with which to transfer cells are disclosed herein.

Recombinant cells of the present invention can also be co-transformed with one or more recombinant molecules including flea synaptic vesicle nucleic acid molecules encoding one or more proteins of the present invention and one or more other nucleic acid molecules encoding other protective compounds, as disclosed herein (e.g., to produce multivalent vaccines).

Recombinant DNA technologies can be used to improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes., addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of all expressed recombinant protein of the present invention may be improved by fragmenting, modifying, or derivatizing nucleic acid molecules encoding such a protein.

Isolated flea synaptic vesicle proteins of the present invention can be produced in a variety of ways, including production and recovery of natural proteins, production and recovery of recombinant proteins, and chemical synthesis of the proteins. In one embodiment, an isolated protein of the present invention is produced by culturing a cell capable of expressing the protein under conditions effective to produce the protein, and recovering the protein. A preferred cell to culture is a recombinant cell of the present invention. Effective culture conditions include, but are not limited to, effective media, bioreactor, temperature, pH and oxygen conditions that permit protein production. An effective, medium refers to any medium in which a cell is cultured to produce a flea synaptic vesicle protein of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

The phrase “recovering the protein”, as well as similar phrases, refers to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization, Proteins of the present invention are preferably retrieved in “substantially pure” form. As used herein, “substantially pure” refers to a purity that allows for the effective use of the protein as a therapeutic composition or diagnostic. A therapeutic composition for animals, for example, should exhibit no substantial toxicity and preferably should be capable of stimulating the production of antibodies in a treated animal.

The present invention also includes isolated (i.e., removed from their natural milieu) antibodies that selectively bind to a flea synaptic vesicle protein of the present invention or a mimetope thereof (e.g., anti-flea synaptic vesicle antibodies). As used herein, the term “selectively binds to” a protein refers to the ability of antibodies of tile present invention to preferentially bind to specified proteins and mimetopes thereof of the present invention. Binding can be measured using a variety of methods standard in the art including enzyme immunoassays (e.g., ELISA), immunoblot assays, etc.; see, for example, Sambrook et al., ibid., and Harlow, et al., 1988, Antibodies, a Laboratory. Manual, Cold Spring Harbor Labs Press; Harlow et al. ibid. An anti-flea synaptic vesicle antibody of the present invention preferably selectively binds to a flea synaptic vesicle protein, respectively, in such a way as to inhibit the function of that protein.

Isolated antibodies of the present invention can include antibodies in serum, o antibodies that have been purified to varying degrees. Antibodies of the present invention can be polyclonal or monoclonal, or can be functional equivalents such as antibody fragments and genetically-engineered antibodies, including single chain antibodies or chimeric antibodies that can bind to one or more epitopes.

A preferred method to produce antibodies of the present invention includes (a) administering to an animal an effective amount of a protein, peptide or mimetope thereof of the present invention to produce the antibodies and (b) recovering the antibodies. In another method, antibodies of the present invention are produced recombinantly using techniques as heretofore disclosed to produce flea synaptic vesicle proteins of the present invention. Antibodies raised against defined proteins or mimetopes can be advantageous because such antibodies are not substantially contaminated with antibodies against other substances that might otherwise cause interference in a diagnostic assay or side effects if used in a therapeutic composition.

Antibodies of the present invention have a variety of potential uses that are within the scope of the present invention. For example, such antibodies can be used (a) as therapeutic compounds to passively immunize an animal in order to protect the animal from fleas susceptible to treatment by such antibodies and/or (b) as tools to screen expression libraries and/or to recover desired proteins of tile present invention from a mixture of proteins and other contaminants. Furthermore, antibodies of the present invention can be used to target cytotoxic agents to fleas in order to directly kill such fleas. Targeting can be accomplished by conjugating (i.e., stably joining) such antibodies to the cytotoxic agents using techniques known to those skilled in the art. Suitable cytotoxic agents are known to those skilled in the art.

One embodiment of the present invention is a therapeutic composition that, when administered to an animal susceptible to flea infestation, is capable of protecting that animal from flea infestation. Therapeutic compositions of the present invention include at least one of the following protective molecules: an isolated flea synaptic vesicle protein; a mimetope of an isolated flea synaptic vesicle protein; an isolated flea synaptic vesicle nucleic acid molecule; and/or a compound derived from said isolated flea synaptic vesicle protein that inhibits flea synaptic vesicle protein activity. A therapeutic composition of the present invention can further comprise a component selected from the group of an excipient, a carrier, and/or an adjuvant; these components are described further herein. As used herein, a protective molecule or protective compound refers to a compound that, when administered to an animal in an effective manner, is able to treat, ameliorate, and/or prevent flea infestation. Preferred fleas to target are heretofore disclosed. One example of a protective molecule is a vaccine, such as, but not limited to, a naked nucleic acid vaccine, a recombinant virus vaccine, a recombinant cell vaccine, and a recombinant protein vaccine. Another example of a protective molecule is a compound that inhibits flea synaptic vesicle protein activity, such as an isolated antibody that selectively binds to a flea synaptic vesicle protein, a substrate analog of a flea synaptic vesicle protein, anti-sense-, triplex formation-, ribozyme-, and/or RNA drug-based compounds, or other inorganic or organic molecules that inhibit flea synaptic vesicle protein activity. Inhibiting flea synaptic vesicle protein activity can refer to the ability of a compound to reduce the activity of flea synaptic vesicle proteins. Inhibiting flea synaptic vesicle protein activity can also refer to the ability of a compound to reduce the amount of flea synaptic vesicle protein in a flea.

One embodiment of the present invention is a therapeutic composition comprising an excipient and a compound selected from tile group consisting of: (a) an isolated nucleic acid molecule selected from the group consisting of a flea cDNA molecule and a flea mRNA molecule, wherein said nucleic acid molecule is at least 30 nucleotides in length and hybridizes with a nucleic acid molecule having a nucleic acid sequence selected from the group consisting of SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and SEQ ID NO:12, under conditions comprising (I) hybridizing in a solution comprising 1×SSC in the absence of nucleic acid helix destabilizing compounds, at a temperature of 37° C. and (2) washing in a solution comprising 1×SSC in the absence of helix destabilizing compounds, at a temperature of 47° C.; (b) an isolated protein encoded by a nucleic acid molecule at least 30 nucleotides in length that hybridizes with a nucleic acid molecule having a nucleic acid sequence selected from the group consisting of SEQ ID NO:4, SEQ ID NO:6, SEQ ID NO:10, and/or SEQ ID NO:12, under conditions comprising (i) hybridizing in a solution comprising 1×SSC in the absence of helix destabilizing compounds, at a temperature of 37° C. and (ii) washing in a solution comprising 1×SSC in the absence of helix destabilizing compounds, at a temperature of 47° C.; and (c) an isolated antibody that selectively binds to a protein of (b).

Another embodiment of the present invention includes a method to reduce flea infestation in an animal susceptible to flea infestation. Such a method includes the step of administering to the animal a therapeutic molecule comprising a protective compound selected from the group consisting, of (a) an isolated flea synaptic vesicle protein; (b) a mimetope of an isolated flea synaptic vesicle protein; (c) an isolated flea synaptic vesicle nucleic acid molecule; and (d) a compound derived from an isolated flea synaptic vesicle protein that inhibits flea synaptic vesicle protein activity.

Therapeutic compositions of the present invention can be administered to any animal susceptible to flea infestation, preferably to mammals, and more preferably to dogs, cats, humans, ferrets, horses, cattle, sheep, and other pets, economic food animals, work animals and/or zoo animals. Preferred animals to protect against flea infestation include dogs, cats, humans, and ferrets, with dogs and cats being particularly preferred.

As used herein, the term derived, or the ter-m derived from, refers to a peptide, antibody, mimetope, nucleic acid molecule, or other compound that was obtained from or obtained using a flea synaptic vesicle protein or nucleic acid molecule of the present invention. Methods to obtain derivatives from a flea synaptic vesicle molecule of the present invention are known in the art, and as such include, but are not limited to molecular modeling of flea synaptic vesicle proteins to determine active sites, and predicting from these active sites smaller fragments and/or mimetopes that retain and/or mimic these active sites, thereby inhibiting flea synaptic vesicle protein activity. Other inhibitors of flea synaptic vesicle activity can also be obtained in a variety of ways, including but not limited to screening of peptide or small chemical compound libraries against flea synaptic vesicle proteins of the present invention; and screening of polyclonal or monoclonal antibodies to find antibodies that specifically bind flea synaptic vesicle proteins of the present invention.

A flea synaptic vesicle protein inhibitor of the present invention (i.e. an inhibitor of a flea synaptic vesicle protein) is identified by its ability to mimic, bind to, modify, or otherwise interact with, a flea synaptic vesicle protein, thereby inhibiting the activity of a natural flea synaptic vesicle protein. Suitable inhibitors of flea synaptic vesicle protein activity are compounds that inhibit flea synaptic vesicle protein activity in at least one of a variety of ways: (a) by binding to or otherwise interacting with or otherwise modifying flea synaptic vesicle protein sites; (b) by binding to the flea synaptic vesicle protein and thus reducing the availability of the flea synaptic vesicle protein in solution; (c) by mimicking a flea synaptic vesicle protein; and (d) by inter-acting with other regions of the flea synaptic vesicle protein to inhibit flea synaptic vesicle protein activity, for example, by allosteric interaction.

Flea synaptic vesicle protein inhibitor-s can be used directly as compounds in compositions of the present invention to treat animals as long as such compounds are not harmful to host animals being treated. Preferred flea synaptic vesicle protein inhibitors of the present invention include, but are not limited to, flea synaptic vesicle protein substrate analogs, and other molecules that bind to a flea synaptic vesicle protein (e.g., to an allosteric site) in such a manner that the activity of the flea synaptic vesicle protein is inhibited. A flea synaptic vesicle protein substrate analog refers to a compound that interacts with (e.g., binds to, associates with, modifies) the active site of a flea synaptic vesicle protein. A preferred flea synaptic vesicle protein substrate analog inhibits flea synaptic vesicle protein activity. Flea synaptic vesicle protein substrate analogs can be of any inorganic or organic composition. Flea synaptic vesicle protein substrate analogs can be, but need not be, structurally similar to a flea synaptic vesicle protein natural substrate as long as they can interact with the active site of that flea synaptic vesicle protein. Flea synaptic vesicle protein substrate analogs can be designed using computer-generated structures of flea synaptic vesicle proteins of the present invention or computer structures of flea synaptic vesicle protein's natural substrates. Preferred sites to model include one or more of the active sites of flea synaptic vesicle proteins. Substrate analogs can also be obtained by generating random samples of molecules, such as oligonucleotides, peptides, peptidomimetic compounds, or other inorganic or organic molecules, and screening such samples for their ability to interfere with interaction between flea synaptic vesicle proteins and their substrates, e.g. by affinity chromatography techniques. A preferred flea synaptic vesicle protein substrate analog is a flea synaptic vesicle protein mimetic compound, i.e., a compound that is structurally and/or Functionally similar to a natural substrate of a flea synaptic vesicle protein of the present invention, particularly to the region of the substrate that interacts with the flea synaptic vesicle protein active site, but that inhibits flea synaptic vesicle protein activity upon interacting with the flea synaptic vesicle protein active site.

The present invention also includes a therapeutic composition comprising at least one protective molecule of the present invention in combination with at least one additional compound protective against one or more infectious agents.

In one embodiment, a therapeutic composition of the present invention can be used to protect an animal from flea infestation by administering such composition to a flea in order to prevent infestation. Such administration to the flea and/or animal could be oral, or by application to the animal's body surface (e.g. topical spot-on, or spraying onto the animal), or by application to the environment (e.g., spraying). Examples of such compositions include, but are not limited to, transgenic vectors capable of producing at least onic therapeutic composition of the present invention. In another embodiment a flea can ingest therapeutic compositions, or products thereof, present on the surface of or in the blood of a host animal that has been administered a therapeutic composition of the present invention.

In accordance with the present invention, a host animal (i.e., an animal that is or is capable of being infested with fleas) is treated by administering to the animal a therapeutic composition of the present invention in such a manner that the composition itself (e.g., a flea synaptic vesicle protein, a flea synaptic vesicle nucleic acid molecule, a flea synaptic vesicle protein inhibitor, a synaptic vesicle protein synthesis suppressor (i.e., a compound that decreases the production or half-life of a synaptic vesicle protein in fleas), a flea synaptic vesicle protein mimetope, or a anti-flea synaptic vesicle antibody) or a product generated by the animal in response to administration of the composition (e.g., antibodies produced in response to administration of a flea synaptic vesicle protein or nucleic acid molecule, or conversion of an inactive inhibitor “prodrug” to an active flea synaptic vesicle protein inhibitor) ultimately enters the flea. A host animal is preferably treated in such a way that the compound or product thereof is present on the body surface of the animal or enters the blood stream of the animal. Fleas are then exposed to the composition or product when they feed from the animal. For example, flea synaptic vesicle protein inhibitors administered to an animal are administered in such a way that the inhibitors enter the blood stream of the animal, where they can be taken up by feeding fleas.

The present invention also includes the ability to reduce larval flea infestation in that when fleas feed from a host animal that has been administered a therapeutic composition of the present invention, at least a portion of compounds of the present invention, or products thereof in the blood taken up by the fleas are excreted by the fleas in feces, which is subsequently ingested by flea larvae. In particular, it is of note that flea larvae obtain most, if not all, of their nutrition from flea feces.

In accordance with the present invention, reducing flea synaptic vesicle protein activity in a flea call lead to a number of outcomes that reduce flea burden on treated animals and their surrounding environments. Such outcomes include, but are not limited to, (a) reducing the viability of fleas that feed from the treated animal, (b) reducing the fecundity of female fleas that feed from the treated animal, (c) reducing the reproductive capacity of male fleas that feed from the treated animal, (d) reducing the viability of eggs laid by female fleas that feed from the treated animal, (e) altering the blood feeding behavior of fleas that feed from the treated animal (e.g., fleas take up less volume per feeding or feed less frequently), (f) reducing the viability of flea larvae, for example due to the feeding of larvae from feces of fleas that feed from the treated animal, (g) altering the development of flea larvae (e.g., by decreasing feeding behavior, inhibiting growth, inhibiting (e.g., slowing or blocking) molting, and/or otherwise inhibiting maturation to adults), and/or (h) altering or decreasing the ability of fleas or flea larvae to digest a blood meal.

In order to protect an animal from flea infestation, a therapeutic composition of the present invention is administered to the animal in an effective manner such that the composition is capable of protecting that animal from flea infestation. Therapeutic compositions of the present invention can be administered to animals prior to infestation in order to prevent infestation (i.e., as a preventative vaccine) and/or can be administered to animals after infestation. For example, proteins, mimetopes thereof, and antibodies thereof can be used as immunotherapeutic agents.

Therapeutic compositions of the present invention can be formulated in an excipient that the animal to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextrin. Excipients can also contain minor amounts of additives, such as substances that enhance isotonicity and chemical stability. Examples of buffers include phosphate buffer, bicarbonate buffer and Tris buffer, while examples of preservatives include thimerosal, or o-cresol, formalin and benzyl alcohol. Standard formulations call either be liquid injectables or solids which can be taken up in a suitable liquid as a suspension or solution for injection. Thus, in a non-liquid formulation, the excipient can comprise dextrose, serum albumin, preservatives, etc., to which sterile water or saline can be added prior to administration.

In one embodiment of the present invention, a therapeutic composition call include an adjuvant. Adjuvants are agents that are capable of enhancing the immune response of an animal to a specific antigen. Suitable adjuvants include, but are not limited to, cytokines, chemokines, and compounds that induce the production of cytokines and chemokines (e.g., granulocyte macrophage colony stimulating factor (GM-CSF), Flt-3 ligand, granulocyte colony stimulating factor (G-CSF), macrophage colony stimulating factor (M-CSF), colony stimulating factor (CSF), erythropoietin (EPO), interleukin 2 (IL-2), interleukin-3 (IL-3), interleukin 4 (IL-4), interleukin 5 (IL-5), interleukin 6 (IL-6), interleukin 7 (L-7), interleukin 8 (IL-8), interleukin 10 (IL-10), interleukin 12 (IL-12), interferon gamma, interferon gamma inducing factor I (IGIF), transforming growth factor beta, RANTES (regulated upon activation, normal T cell expressed and presumably secreted), macrophage inflammatory proteins (e.g., MIP-1 alpha and MIP-1 beta), and Leishmania elongation initiating factor (LEIF)); bacterial components (e.g., endotoxins, in particular superantigens, exotoxins and cell wall components); aluminum-based salts, calcium-based salts; silica; polynucleotides; toxoids; serum proteins, viral coat proteins: block copolymer adjuvants (e.g., Hunter's Titermax™ adjuvant (Vaxcel™, Inc. Norcross, Ga.), Ribi adjuvants (Ribi ImmunoChem Research, Inc., Hamilton, Mont.); and saponins and their derivatives (e.g., Quil A (Superfos Biosector A/S, Denmark). Protein adjuvants of the present invention can be delivered in the form of the protein themselves or of nucleic acid molecules encoding such proteins using the methods described herein.

In one embodiment of the present invention, a therapeutic composition can include a carrier. Carriers include compounds that increase the half-life of a therapeutic composition in the treated animal. Suitable carriers include, but are not limited to, polymeric controlled release vehicles, biodegradable implants, liposomes, bacteria, viruses, other cells, oils, esters, and glycols.

One embodiment of thc present invention is a controlled release formulation that is capable of slowly releasing, a composition of the present invention into an animal. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

A preferred controlled release formulation of the present invention is capable of releasing a composition of the present invention into the blood of the treated animal at a constant rate sufficient to attain therapeutic dose levels of the composition. The therapeutic composition is preferably released over a period of time ranging from 1 to 12 months. A controlled release formulation of the present invention is capable of effecting a treatment preferably for at least 1 month, preferably for at least 3 months, preferably for at least 6 months, preferably for at least 9 months, and preferably for at least 12 months.

Acceptable protocols to administer therapeutic compositions in an effective manner include individual dose size, number of doses, frequency of dose administration, and mode of administration. Determination of such protocols can be accomplished by those skilled in the art. A suitable single dose is a dose that is capable of treating an animal when administered one or more times over a suitable time period. For example, a preferred single dose of an inhibitor is from about 1 microgram (μg) to about 10 milligrams (mg) of the therapeutic composition per kilogram body weight of the animal. Booster vaccinations can be administered from about 2 weeks to several years after the original administration. Booster administrations preferably arc administered when the immune response of the animal becomes insufficient to protect the animal from disease. A preferred administration schedule is one in which from about 10 μg to about 1 mg of the therapeutic composition per kg body sleight of the animal is administered from about one to about two times over a time period of from about 2 weeks to about 12 months. Modes of administration can include, but are not limited to, subcutaneous, intradermal, intravenous, intranasal, oral, transdermal, intraocular, intranasal, conjunctival, and intramuscular routes. Methods of administration for other therapeutic compounds can be determined by one skilled in the art, and nay include administration of a therapeutic composition one or more times, on a daily, weekly, monthly or yearly regimen; routes of administration can be determined by one skilled in the art, and may include any route. A preferred route of administration of an inhibitory compound when administering to fleas is a topical, or “spot-on” formulation administered to the body surface of the animal, so that a flea would encounter the inhibitory compound when attached to the animal; another preferred route of administration of an inhibitory compound is an oral formulation that, when fed to an animal, would enter the bloodstream of the animal, which would then be transferred to a flea while feeding from the animal.

A recombinant protein vaccine of the present invention comprises a recombinantly-produced flea synaptic vesicle protein of the present invention that is administered to an animal according to a protocol that results in the animal producing a sufficient immune response to protect itself from a flea infestation. Such protocols can be determined by those skilled in the art.

According to one embodiment, a nucleic acid molecule of the present invention can be administered to an animal in a fashion to enable expression of that nucleic acid molecule into a protective protein or protective RNA (e.g., antisense RNA, ribozyme, triple helix forms or RNA drug) in the animal. Nucleic acid molecules can be delivered to an animal in a variety of methods including, but not limited to, (a) administering a naked (i.e., not packaged in a viral coat or cellular membrane) nucleic acid as a genetic vaccine (e.g., as naked DNA or RNA molecules, such as is taught, for example in Wolff et al., 1990, Science 247. 1465-1468) or (b) administering a nucleic acid molecule packaged as a recombinant virus vaccine or as a recombinant cell vaccine (i.e., the nucleic acid molecule is delivered by a viral or cellular vehicle).

A genetic (i.e., naked nucleic acid) vaccine of tie present invention includes a nucleic acid molecule of the present invention and preferably includes a recombinant molecule of the present invention that preferably is replication, or otherwise amplifications competent. A genetic vaccine of the present invention can comprise one or more nucleic acid molecules of the present invention in the form of, for example, a dicistronic recombinant molecule. Preferred genetic vaccines include at least a portion of a viral genome, i.e., a viral vector. Prefer-red viral vectors include those based on alphaviruses, poxviruses, adenoviruses, herpesviruses, picornaviruses, and retroviruses, with those based on alphaviruses, such as sindbis or Semliki forest virus, species-specific herpesviruses and poxviruses being particularly preferred. Any suitable transcription control sequence can be used, including those disclosed as suitable for protein production. Particularly preferred transcription control sequences include cytomegalovirus immediate early (preferably in conjunction with Intron-A), Rous sarcoma virus long terminal repeat, and tissue-specific transcription control sequences, as well as transcription control sequences endogenous to viral vectors if viral vectors are used. The incorporation of a “strong” polyadenylation signal is also preferred.

Genetic vaccines of the present invention can be administered in a variety of ways, with intramuscular, subcutaneous, intradermal, transdermal, conjunctival, intraocular, intranasal and oral routes of administration being preferred. A preferred single dose of a genetic vaccine ranges from about 1 nanogram (ng) to about 600 μg, depending on the route of administration and/or method of delivery, as can be determined by those skilled in the art. Suitable delivery methods include, for example, by injection, as drops, aerosolized and/or topically. Genetic vaccines of the present invention can be contained in an aqueous excipient (e.g., phosphate buffered saline) alone or in a carrier (e.g., lipid-based vehicles).

A recombinant virus vaccine of the present invention includes a recombinant molecule of the present invention that is packaged in a viral coat and that can be expressed in an animal after administration. Preferably, the recombinant molecule is packaging- or replication-deficient and/or encodes (in attenuated virus. A number of recombinant viruses can be used, including, but not limited to, those based on alplhaviruses, poxviruses, adenoviruses, herpesviruses, picornaviruses, and retroviruses. Preferred recombinant virus vaccines are those based on alphaviruses (such as Sindbis virus), raccoon poxviruses, species-specific herpesviruses and species-specific poxviruses. An example of methods to produce and use alphavirus recombinant virus vaccines are disclosed in U.S. Pat. No. 5,766,602 to Xiong, and Grieve.

When administered to an animal, a recombinant virus vaccine of the present invention infects cells within the immunized animal and directs the production of a protective protein or RNA nucleic acid molecule that is capable of protecting the animal from flea infestation as disclosed herein. For example, a recombinant virus vaccine comprising a flea synaptic vesicle nucleic acid molecule of the present invention is administered according to a protocol that results in the animal producing a sufficient immune response to protect itself from flea infestation. A preferred single dose of a recombinant virus vaccine of the present invention is from about 1×10⁴ to about 1×10⁸ virus plaque forming units (pfu) per kilogram body weight of the animal. Administration protocols are similar to those described herein for protein-based vaccines, with subcutaneous, intramuscular, intranasal, intraocular, conjunctival, and oral administration routes being preferred.

A recombinant cell vaccine of the present invention includes recombinant cells of the present invention that express at least one protein of the present invention. Preferred recombinant cells for this embodiment include Salmonella, E, coli, Listeria, Mycobacterium, S. frugiperda, yeast, (including Saccharomyces cerevisiae and Pichia pastoris), BHK, CV-1, myoblast G8, COS (e.g., COS-7), Vero, MDCK and CRFK recombinant cells. Recombinant cell vaccines of the present invention can be administered in a variety of ways but have the advantage that they can be administered orally, preferably at doses ranging from about 10⁸ to about 10¹² cells per kilogram body weight. Administration protocols are similar to those described herein for protein-based vaccines. Recombinant cell vaccines can comprise whole cells, cells stripped of cell walls or cell lysates.

The efficacy of a therapeutic composition of the present invention to protect an animal from flea infestation can be tested in a variety of ways including, but not limited to, detection of protective antibodies (using, for example, proteins or mimetopes of the present invention), detection of cellular immunity within tile treated animal, or challenge of the treated animal with the fleas to determine whether the treated animal is resistant to infestation. Challenge studies call include direct administration of fleas to the treated animal. In one embodiment, therapeutic compositions call be tested in animal models such as mice. Such techniques are known to those skilled in the art.

As discussed herein, one therapeutic composition of the present invention includes an inhibitor of flea synaptic vesicle protein activity, i.e., a compound capable of substantially interfering with the function of a flea synaptic vesicle protein. An inhibitor of flea synaptic vesicle protein activity, or function, can be identified using flea synaptic vesicle proteins of the present invention. A preferred inhibitor of flea synaptic vesicle protein function is a compound capable of substantially interfering with the function of a flea synaptic vesicle protein and which does not substantially interfere with the function of host animal synaptic vesicle proteins. As used herein, a compound that does not substantially inhibit or interfere with host animal synaptic vesicle proteins is one that, when administered to a host animal, tile host animal shows no significant adverse effects attributable to the inhibition of synaptic vesicle and which, when administered to an animal in an effective manner, is capable of protecting that animal from flea infestation.

One embodiment of the present invention is a method to identify a compound capable of inhibiting flea synaptic vesicle protein activity. Such a method includes the steps of (a) contacting (e.g., combining, mixing) an isolated flea synaptic vesicle protein of the present invention, with a putative inhibitory compound under conditions in which, in the absence of the compound, the protein has flea synaptic vesicle protein activity, and (b) determining if the putative inhibitory compound inhibits the activity. Flea synaptic vesicle protein activity can be determined in a variety of ways known in the art, including but not limited to determining the ability of flea synaptic vesicle protein to bind to or otherwise interact with a substrate. Such conditions under which a flea synaptic vesicle protein has flea synaptic vesicle protein activity include conditions in which a flea synaptic vesicle protein has a correct three-dimensionally folded structure under physiologic conditions, i.e. physiologic pH, physiologic ionic concentrations, and physiologic temperatures.

Putative inhibitory compounds to screen include antibodies (including fragments and mimetopes thereof), putative substrate analogs, and other, preferably small, organic or inorganic molecules. Methods to determine flea synaptic vesicle protein activity are known to those skilled in the art.

A preferred method to identify a compound capable of inhibiting flea synaptic vesicle protein activity includes contacting an isolated flea synaptic vesicle protein of the present invention with a putative inhibitory compound under conditions in which, in the absence of the compound, the protein has flea synaptic vesicle protein activity; and determining if the putative inhibitory compound inhibits the activity.

Another embodiment of the present invention is an assay kit to identify an inhibitor of a flea synaptic vesicle protein of the present invention. This kit comprises an isolated flea synaptic vesicle protein of the present invention, and a means for determining inhibition of an activity of flea synaptic vesicle protein, where the means enables detection of inhibition. Detection of inhibition of flea synaptic vesicle protein identifies a putative inhibitor to be an inhibitor of a flea synaptic vesicle protein. Means for determining inhibition of a flea synaptic vesicle protein include, for example, an assay system that detects binding of a putative inhibitor to a flea synaptic vesicle molecule, and an assay system that detects interference by a putative inhibitor of the ability of flea synaptic vesicle protein to hydrolyze a substrate. Means and methods are described herein and are known to those skilled in the art.

The following examples are provided for the purposes of illustration and are not intended to limit the scope of the present invention. The following examples include a number of recombinant DNA and protein chemistry techniques known to those skilled in the art; see, for example, Sambrook et al., ibid.

### Example 1

This Example describes the isolation of RNA from the hindgut and Malpighian tubules (HMT) of Ctenocephalides felis and the use of isolated RNA to construct subtracted and unsubtracted cDNA libraries.

Approximately 10,000 hindguts and Malpighian tubules were dissected from equal numbers of cat blood fed and unfed adult C. felis with a male to female ratio of 1 to 4, and total RNA was extracted using a guanidine isothiocyanate lysis buffer and the standard procedure described by Sambrook et al. Poly-A enriched mRNA was purified from total RNA above using a mRNA Purification Kit, available from Pharmacia Biotech, Piscataway, N. J., following, the manufacturer's protocol. The same procedures ere used to extract total RNA and isolate poly-A enriched mRNA from the dissected C. felis bodies following removal of HMT, referred to hereinafter as “non-HMT mRNA”.

Poly-A enriched mRNA was used to construct a cDNA library using subtractive hybridization and suppression PCR as follows. Subtractive hybridization and suppression PCR was conducted using a PCR-Select™ cDNA Subtraction Kit, available from Clontech Laboratories, Inc., Palo Alto, Calif. according to the manufacturer's instructions. Briefly, this kit uses subtractive hybridization and suppression PCR to specifically amplify cDNA sequences that are present in the tester cDNA and absent in the driver cDNA, thus clinching for tester-specific sequences. The efficiency of the subtraction process can be assessed by semi-quantitative PCR and by comparing the ethidium bromide staining patterns of the subtracted and unsubtracted samples on agarose gels as described in section V. D. of the manufacturer's protocol. For the semi-quantitative PCR, three genes with mRNAs known to be expressed outside of the HMT tissue were used to test for specific subtraction. These genes encoded putative actin, N-aminopeptidase, and serine protease proteins.

Subtractive hybridization and suppression PCR was conducted under the following conditions. Two micrograms (μg) of HMT mRNA was used as the template for synthesis of the tester material and 2 μg of non-HMT mRNA was used as template for synthesis of the driver material in this reaction. The number of cycles used in the selective amplification steps was optimized using the manufacturer's protocols. Optimization resulted in the use of 24 rather than the standard 27 cycles of primary PCR in combination with 15 cycles of secondary PCR rather than the standard 12 cycles.

The products from the suppressive PCR reaction were ligated into the pCR®2.1 vector, available from Invitrogen, Carlsbad, Calif., using an Original TA Cloning® Kit, available from Invitrogen. The ligation reaction was then used to transform INVαF One Shot™ competent cells, available from Invitrogen, which were plated on Luria broth (LB) agar with 50 micrograms per milliliter (μg/l) ampicillin, available from Sigma-Aldrich Co. St. Louis, Mo., and 50 μg/ml 5-bromo-4-chloro-3-indoyl β-D-galactopyranoside (X-Gal), available from Fisher Biotech, Fair Lawn, N.J. Transformed colonies were amplified and the DNA isolated using the standard alkaline lysis procedure described by Sambrook et al., ibid.

Automated cycle sequencing of DNA samples was performed using all ABI PRISM™ Model 377, available from Perkins Elmer, with XL upgrade DNA Sequencer, available from PE Applied Biosystems, Foster City, Calif., after reactions were carried out using the PRISM™ Dye Terminator Cycle Sequencing Ready Reaction Kit or the PRISM™ dRhodamine Terminator Cycle Sequencing Ready Reaction Kit or the PRISM™ BigDye™ Terminator Cycle sequencing Ready Reaction Kit, available from PE Applied Biosystems, following the manufacturer's protocol, hereinafter “standard sequencing methods”. Sequence analysis was performed using SeqLab, using default parameters. Each sequence read was trimmed of vector sequence at either end and submitted for a search through the National Center for Biotechnology Information (NCBI), National Library of Medicine, National Institute of Health, Baltimore, Md., using the BLAST network. This database includes SwissProt+PIR+SPupdate+GenPept+GPUpdate+PDB databases. The search was conducted using the xBLAST function, which compares the translated sequences in all 6 reading frames to the protein sequences contained in the database.

An unsubtracted HMT cDNA library was constructed as follows. Approximately 10,000 HMT tissues were dissected from equal numbers of unfed and cat blood-fed adult C. felis with a male to female ratio of 1:4. Total RNA was extracted using a guanidine isothiocyanate lysis buffer and procedures described in Sambrook et al., followed by isolation using a mRNA purification kit, available from Pharmacia, according to the manufacturer's protocols. The library was constructed with 5 μg of isolated mRNA using a ZAP-cDNA®cDNA synthesis kit, and packaged using a ZAP-cDNA®Gigapack® gold cloning kit, both available from Stratagene, La Jolla, Calif. The resultant HMT library was amplified to a titer of about 5×10⁹ plaque forming units per milliliter (pfu/ml). Single clone excisions were performed using the Ex-Assist™ helper phage, available from Stratagene, and used to create double stranded plasmid template for sequencing using the manufacturer's protocols with the following exceptions. Following incubation of the SOLR cells with the cleared phage lysate, the mixture was used to inoculate LB broth, and the mix was incubated overnight and then subjected to mini-prep plasmid preparation and sequencing as described for the subtracted HMT library above.

### Example 2

This Example describes the further characterization of synaptic vesicle 2B-like sequence nucleic acid molecules of the present invention.

A cDNA, designated clone 2104-59, was isolated from the subtracted HMT library as described in Example 1; the nucleic acid sequence of the cDNA's coding strand is denoted herein as SEQ ID NO:1. DNA from clone 2104-59 was purified, and the insert used for plaque hybridization screening of the unsubtracted HMT cDNA library as follows. The insert from clone 2104-59 was excised by digestion with EcoRI, separated by agarose gel electrophoresis and purified using the QiaQuick Gel Extraction kit, available from Qiagen. A Megaprime DNA labeling kit, available from Amersham Pharmacia, was used to incorporate α-³²P-labeled dATP into the random-primed probe mix. The ³²P α-dATP labeled probe was used in a plaque lift hybridization as follows. Filters were hybridized with about 1×10⁶ counts per minute (cpm) per ml of tile probe in 5×SSPE, (see Sambrook et al., ibid.), 1.2% sodium dodecyl sulfate (SDS), 0.1 mg/ml salmon sperm DNA and 5× Denhardt's reagent, (see Sambrook et al., ibid.), at 55° C. for about 14 hours. The filters were washed as follows: (a) 10 minutes with 5×SSPE and 1% SDS, (b) 10 minutes with 2×SSPE and 1% SDS, (c) 10 minutes with 1×SSPE and 0.5% SDS, and (d) 10 minutes with 0.5×SSPE and 1% SDS. All washes were conducted at 55° C. Plaques that hybridized strongly to the probe were isolated and subjected to in vivo excision. In vivo excision was performed using the Stratagene Ex-Assist™ helper phage system and protocols, available from Stratagene, to convert a positive plaque to pBluescript™ plasmid DNA. Sequencing was conducted using standard sequencing methods following preparation of DNA with a Qiagen Qiaprep™ spin mini prep kit, available from Qiagen, using the manufacturer's instructions and restriction enzyme digestion with about 1 μl of 20 U/μl each of EcoRI and XhoI, available from New England Biolabs, Beverly, Mass.

Hybridization and plaque purification resulted in the isolation of a clone containing, an about 1875 nucleotide synaptic vesicle 2B-like sequence, referred to herein as nCfSVP1₁₈₇₅, with a coding strand denoted nucleic acid sequence SEQ ID NO:2 and a complementary sequence denoted SEQ ID NO:4. Translation of SEQ ID NO:2 suggests that nucleic acid molecule nCfSVP1₁₈₇₅ encodes a full-length synaptic vesicle 2B-like protein of 530 amino acids, referred to herein as PCfSVP1₅₃₀ with an amino acid sequence represented by SEQ ID NO:3, assuming the initiation codon spans from nucleotide 44 through nucleotide 46 of SEQ ID NO:2 and the termination codon spans from nucleotide 1634 through nucleotide 1636 of SEQ ID NO:2. The coding region encoding PCfSVP1₅₃₀, is represented by nucleic acid molecule nCfSVP1₁₅₉₀, with a coding strand with the nucleic acid sequence denoted SEQ ID NO:5 and a complementary strand with nucleic acid sequence denoted SEQ ID NO:6. The amino acid sequence of SEQ ID NO:3, predicts that PCfSVP1₅₃₀ has an estimated molecular weight of about 58.7 kDa and an isoelectric point (pI) of about 7.61.

Comparison of amino acid sequence SEQ ID NO:3 with amino acid sequences reported in GenBank indicates that SEQ ID NO:3 showed the most homology, i.e., about 32% identity, with a Drosophila melanogaster BACR7A4.Y sequence (Accession # CAB51685). Comparison of SEQ ID NO:5 with nucleic acid sequences reported in GenBank indicates that SEQ ID NO:5 showed the most homology, i.e., about 39% identity, with a Rattus norvegicus synaptic vesicle protein 2B (SVP2B) mRNA (Accession # L10362). Percent identity calculations were performed using SeqLab with default parameters.

### Example 3

This Example describes the further characterization and isolation of synaptic vesicle 2B-like sequence nucleic acid molecules of the present invention.

A cDNA designated clone 2089-13 was isolated from the subtracted HMT library as described in Example 1, the nucleic acid sequence of: cDNA's coding strand is denoted herein as SEQ ID NO:7. DNA from clone 2089-13 was purified, and the insert used for plaque hybridization screening of the unsubtracted HMT cDNA library as described in Example 2. Hybridization and plaque purification resulted in the isolation of a clone containing an about 3314 nucleotide synaptic vesicle 2B-like sequence, referred to herein as nCfSVP2₃₃₁₄ with a coding strand denoted nucleic acid sequence SEQ ID NO:8 and a complementary sequence denoted SEQ ID NO:10. Translation of SEQ ID NO:8 suggests that nucleic acid molecule nCrSVP2₃₃₁₄ encodes a full-length synaptic vesicle 2B-like protein of 773 amino acids, referred to herein as PCfSVP2₇₇₃, with an amino acid sequence denoted SEQ ID NO:9, assuming the initiation codon spans from nucleotide 302 through nucleotide 304 of SEQ ID NO:8 and the termination codon spans from nucleotide 2621 through nucleotide 2623 of SEQ ID NO:8. The coding region encoding PCfSVP2₇₇₃, is represented by nucleic acid molecule nCfSVP2₂₃₁₉, with a coding strand denoted SEQ ID NO:11 and a complementary strand denoted SEQ ID NO:12. The amino acid sequence of SEQ ID NO:9, predicts that PCfSVP2₇₇₃ has an estimated molecular weight of about 84.6 kDa and an pt of about 7.1.

Comparison of amino acid sequence SEQ ID NO:9 with amino acid sequences reported in GenBank indicates that SEQ ID NO:9 showed the most homology, i.e., about 68% identity, with a Drosophila melanogaster CG3168 gene product sequence (Accession # AAF46193). Comparison of SEQ ID NO:11 with nucleic acid sequences reported in GenBank indicates that SEQ ID NO:11 showed the most homology, i.e., about 40% identity, with a Homo sapiens KIAA0735 sequence (Accession # NM014848). Percent identity calculations were performed using SeqLab with default parameters.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims:

1. An isolated nucleic acid molecule selected from the group consisting of a flea cDNA molecule and a flea RNA molecule, wherein said nucleic acid molecule is at least 30 nucleotides in length and hybridizes with a nucleic acid molecule having a nucleic acid sequence selected from the group consisting of SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and SEQ ID NO:12, under conditions comprising (a) hybridizing in a solution comprising 1×SSC in the absence of nucleic acid helix destabilizing compounds, at a temperature of 37° C. and (b) washing in a solution comprising 1×SSC in the absence of helix destabilizing compounds, at a temperature of 47° C.

2. The nucleic acid molecule of claim 1, wherein said nucleic acid molecule has a nucleic acid sequence that is at least 70% identical to a nucleic acid sequence selected from the group consisting of SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and SEQ ID NO:12, wherein percentage identity is determined using the Needleman-Wunsch algorithm available in a SeqLab software program, using SeqLab default parameters.

3. The nucleic acid molecule of claim 1, wherein said nucleic acid molecule comprises a nucleic acid sequence at least 30 nucleotides in length identical to a 30 nucleotide portion of a nucleic acid sequence selected from the group consisting of SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and SEQ ID NO:12.

4. The nucleic acid molecule of claim 1, wherein said nucleic acid molecule is selected from the group consisting of: a nucleic acid molecule that encodes a protein having an amino acid sequence selected from the group consisting of SEQ ID NO:3 and SEQ ID NO:9.

5. A recombinant molecule comprising a nucleic acid molecule as set forth in claim 1 operatively linked to a transcription control sequence.

6. A recombinant virus comprising a nucleic acid molecule as set forth in claim 1.

7. A recombinant cell comprising a nucleic acid molecule as set forth in claim 1.

8. A composition comprising an excipient and an isolated nucleic acid molecule of claim 1.

9. (Canceled)

10. The composition of claim 8, further comprising a component selected from the group consisting of an adjuvant and a carrier.

11. A method to produce a protein encoded by an isolated nucleic acid molecule of claim 1, said method comprising culturing a cell transformed with a nucleic acid molecule encoding said protein.

12. The method of claim 11, wherein said protein has an amino acid sequence selected from the group consisting of: SEQ ID NO:3 and SEQ ID NO:9.

13. The method of claim 11, wherein said nucleic acid molecule comprises a nucleic acid sequence selected from the group consisting of: SEQ ID NO:1, SEQ ID NO:2, SEQ ID NO:5, SEQ ID NO:7, SEQ ID NO:8, and/or SEQ ID NO:11.

14. An isolated nucleic acid molecule having a nucleic acid sequence comprising at least 30 nucleotides identical in sequence to a 30 nucleotide portion of a nucleic acid sequence selected from the group consisting of SEQ ID NO:1, SEQ ID NO:2, SEQ ID NO:4, SEQ ID NO:5, SEQ ID NO:6, SEQ ID NO:7, SEQ ID NO:8, SEQ ID NO:10, SEQ ID NO:11, and SEQ ID NO:12.

15. An isolated protein encoded by a nucleic acid molecule selected from the group consisting of: (a) a nucleic acid molecule at least 30 nucleotides in length that hybridizes with a nucleic acid molecule having a nucleic acid sequence selected from the group consisting of SEQ ID NO:4, SEQ ID NO:6, SEQ ID NO:10, and/or SEQ ID NO:12, under conditions comprising (i) hybridizing in a solution comprising 1×SSC in the absence of helix destabilizing compounds, at a temperature of 37° C. and (ii) washing in a solution comprising 1×SSC in the absence of helix destabilizing compounds, at a temperature of 47° C.; and (b) an isolated protein selected from the group consisting of: (1) a protein comprising an amino acid sequence selected from the group consisting of SEQ ID NO:3 and SEQ ID NO:9; and (2) a protein comprising an at least 10 contiguous amino acid portion identical in sequence to an at least 10 contiguous amino acid portion of an amino acid sequence of (1).

16. The protein of claim 15, wherein said protein is encoded by a nucleic acid molecule having a nucleic acid sequence selected from the group consisting of: SEQ ID NO:1, SEQ ID NO:2, SEQ ID NO:5, SEQ ID NO:7, SEQ ED NO:8, and/or SEQ ID NO:11.

17. The protein of claim 15, wherein said protein comprises an amino acid sequence selected from the group consisting of SEQ ID NO:3 and SEQ ID NO:9.

18. A composition comprising an excipient and an isolated protein of claim 15.

19. (Canceled)

20. An isolated antibody that selectively binds to a protein as set forth in claim 15.

21. A composition comprising an excipient and an isolated antibody of claim 20.

22. (Canceled)

23. A method to detect an inhibitor of flea synaptic vesicle activity, said method comprising (a) contacting an isolated flea synaptic vesicle protein of claim 15, with a putative inhibitory compound under conditions in which, in the absence of said compound, said protein has flea synaptic vesicle protein activity, and (b) determining if said putative inhibitory compound inhibits flea synaptic vesicle protein activity.

24. (Canceled)

25. (Canceled)


SEQUENCE LISTING (12 sequences)

SEQ ID NO: 1 - DNA, 498 bp, Ctenocephalides felis
cttggcatctagttttgtaaattcagtttggctgtttattttgctgcgatatttcaatggatttttcgtatctggaggaagtgcaacaatatatgcatatttaggagaatttcataatcctaggcatcgcagcagggctattatgggagcgtcaagcatcttcggatttgcgtgtcttgcattaccgacggttgcatggttaattataaatcagaaatggtcattctatattgactttttgggatatacatacaagccctggaggttgtatatggttgcatgtggtttgccatcactgctttgttgttttgctttgtggaaattaccagaaagtcccaaatttttgatgaatcagggaagaaacgaagaagctcgtcaaattattgccaaaatgtatagaattaatactggtaaaccagaaagtgaattccccgtatcatcaatcttagatgaatatccaggagtggatggtgaaaatacaaataaaacaaagaaatc

SEQ ID NO: 2 - DNA, 1875 bp, Ctenocephalides felis
  CDS (44)..(1633)
acaaaacggtttaattgaatctgtagtttggaaattaataaatatggacagtaacacggggattcaaataatagcttccaaagaaccaaaaccaaggcagtttgaagatgcgttggcactcacaggttttggaaaattcaattaccttcttctggtcgtgagtggatgcgtattagtatgtgttttgatggaaactcttggaatgagttttgtcgttccttcagcacaatgtgatctggaattaacaacaaaacaaaaaggaatattaagcgctatagcttttataggtattataagcagttcacatttatggggatttttagccgatacgagagggaggcggaaagtgattatgcctacacttcttcttgcatttttttgtaccttggcatctagttttgtaaattcagtttggctgtttattttgctgcgatatttcaatggatttttcgtatctggaggaagtgcaacaatatatgcatatttaggagaatttcataatcctaggcatcgcagcagggctattatgggagcgtcaagcatcttcggatttgcgtgtcttgcattaccgacggttgcatggttaattataaatcagaaatggtcattctatattgactttttgggatatacatacaagccctggaggttgtatatggttgcatgtggtttgccatcactgctttgttgttttgctttgtggaaattaccagaaagtcccaaatttttgatgaatcagggaagaaacgaagaagctcgtcaaattattgccaaaatgtatagaattaatactggtaaaccagaaagtgaattccccgtatcatcaatcttagatgaatatccaggagtggatggtgaaaatacaaataaaacaaagaaatcatttttaagaactgtatgggatcaaactgctccgctgtttatgggtgagcacatgaaaaaaacactcattgcatgtactctgcaattcggaatatttgccacatctaacggcatgtacatgtggtttcccgatatcataagtaaaatgacagaatttcaaaacgctcatccaggagtaccaagtacaatatgctacgttgtccaaaattcatcaatgcttagggcggacgacttcattgatactacgaatagtacgaccgagtgtaaagacacaatggaagaacaggcatttatgcattctttaatgttggaagctggatatgcgattggatttcccataataggtgctattattaactctgtgggaaagcttccaattcttgtattcgtgatggtctcatgcgggatttgtggaataatttgtgcatttattgaacatttaacaatagcatcatatttatatttgtggttgctggtttgcggaattgctgtgacagtggtaaacgcggcattagtagatctgtaccctacacaactcagggcaatggcagtatgtatatctttaatgatgggtcgattaggaagtgttgttggaagcaatgttgtaggaataattctagattataattgcgatttaacatttttaatatcaggaacatctctgatagcatgtggagtgattgcattctttataccgaacatttatcagaagcaagtggatagaagaacgagcattgcttcatatggaccataaccatatttccgtcgtgtgttgtgaaatgcaattatactttgagaaagtattatcaattacaaagaaaattcagagttgctactgaatcattttctaaaacatcgacatgaaaattaataactctttattgttattagattcgatgtaagatatatgtacatactcataggtaaaaaatgttaatgccgtcaatgttcaaaattataaagagaataaataaatacgttaaaaaaaaaaaa

SEQ ID NO: 3 - protein, 530 aa, Ctenocephalides felis
MDSNTGIQIIASKEPKPRQFEDALALTGFGKFNYLLLVVSGCVLVCVLMETLGMSFVVPSAQCDLELTTKQKGILSAIAFIGIISSSHLWGFLADTRGRRKVIMPTLLLAFFCTLASSFVNSVWLFILLRYFNGFFVSGGSATIYAYLGEFHNPRHRSRAIMGASSIFGFACLALPTVAWLIINQKWSFYIDFLGYTYKPWRLYMVACGLPSLLCCFALWKLPESPKFLMNQGRNEEARQIIAKMYRINTGKPESEFPVSSILDEYPGVDGENTNKTKKSFLRTVWDQTAPLFMGEHMKKTLIACTLQFGIFATSNGMYMWFPDIISKMTEFQNAHPGVPSTICYVVQNSSMLRADDFIDTTNSTTECKDTMEEQAFMHSLMLEAGYAIGFPIIGAIINSVGKLPILVFVMVSCGICGIICAFIEHLTIASYLYLWLLVCGIAVTVVNAALVDLYPTQLRAMAVCISLMMGRLGSVVGSNVVGIILDYNCDLTFLISGTSLIACGVIAFFIPNIYQKQVDRRTSIASYGP

SEQ ID NO: 4 - DNA, 1875 bp, Ctenocephalides felis
ttttttttttttaacgtatttatttattctctttataattttgaacattgacggcattaacattttttacctatgagtatgtacatatatcttacatcgaatctaataacaataaagagttattaattttcatgtcgatgttttagaaaatgattcagtagcaactctgaattttctttgtaattgataatactttctcaaagtataattgcatttcacaacacacgacggaaatatggttatggtccatatgaagcaatgctcgttcttctatccacttgcttctgataaatgttcggtataaagaatgcaatcactccacatgctatcagagatgttcctgatattaaaaatgttaaatcgcaattataatctagaattattcctacaacattgcttccaacaacacttcctaatcgacccatcattaaagatatacatactgccattgccctgagttgtgtagggtacagatctactaatgccgcgtttaccactgtcacagcaattccgcaaaccagcaaccacaaatataaatatgatgctattgttaaatgttcaataaatgcacaaattattccacaaatcccgcatgagaccatcacgaatacaagaattggaagctttcccacagagttaataatagcacctattatgggaaatccaatcgcatatccagcttccaacattaaagaatgcataaatgcctgttcttccattgtgtctttacactcggtcgtactattcgtagtatcaatgaagtcgtccgccctaagcattgatgaattttggacaacgtagcatattgtacttggtactcctggatgagcgttttgaaattctgtcattttacttatgatatcgggaaaccacatgtacatgccgttagatgtggcaaatattccgaattgcagagtacatgcaatgagtgtttttttcatgtgctcacccataaacagcggagcagtttgatcccatacagttcttaaaaatgatttctttgttttatttgtattttcaccatccactcctggatattcatctaagattgatgatacggggaattcactttctggtttaccagtattaattctatacattttggcaataatttgacgagcttcttcgtttcttccctgattcatcaaaaatttgggactttctggtaatttccacaaagcaaaacaacaaagcagtgatggcaaaccacatgcaaccatatacaacctccagggcttgtatgtatatcccaaaaagtcaatatagaatgaccatttctgatttataattaaccatgcaaccgtcggtaatgcaagacacgcaaatccgaagatgcttgacgctcccataatagccctgctgcgatgcctaggattatgaaattctcctaaatatgcatatattgttgcacttcctccagatacgaaaaatccattgaaatatcgcagcaaaataaacagccaaactgaatttacaaaactagatgccaaggtacaaaaaaatgcaagaagaagtgtaggcataatcactttccgcctccctctcgtatcggctaaaaatccccataaatgtgaactgcttataatacctataaaagctatagcgcttaatattcctttttgttttgttgttaattccagatcacattgtgctgaaggaacgacaaaactcattccaagagtttccatcaaaacacatactaatacgcatccactcacgaccagaagaaggtaattgaattttccaaaacctgtgagtgccaacgcatcttcaaactgccttggttttggttctttggaagctattatttgaatccccgtgttactgtccatatttattaatttccaaactacagattcaattaaaccgttttgt

SEQ ID NO: 5 - DNA, 1590 bp, Ctenocephalides felis
atggacagtaacacggggattcaaataatagcttccaaagaaccaaaaccaaggcagtttgaagatgcgttggcactcacaggttttggaaaattcaattaccttcttctggtcgtgagtggatgcgtattagtatgtgttttgatggaaactcttggaatgagttttgtcgttccttcagcacaatgtgatctggaattaacaacaaaacaaaaaggaatattaagcgctatagcttttataggtattataagcagttcacatttatggggatttttagccgatacgagagggaggcggaaagtgattatgcctacacttcttcttgcatttttttgtaccttggcatctagttttgtaaattcagtttggctgtttattttgctgcgatatttcaatggatttttcgtatctggaggaagtgcaacaatatatgcatatttaggagaatttcataatcctaggcatcgcagcagggctattatgggagcgtcaagcatcttcggatttgcgtgtcttgcattaccgacggttgcatggttaattataaatcagaaatggtcattctatattgactttttgggatatacatacaagccctggaggttgtatatggttgcatgtggtttgccatcactgctttgttgttttgctttgtggaaattaccagaaagtcccaaatttttgatgaatcagggaagaaacgaagaagctcgtcaaattattgccaaaatgtatagaattaatactggtaaaccagaaagtgaattccccgtatcatcaatcttagatgaatatccaggagtggatggtgaaaatacaaataaaacaaagaaatcatttttaagaactgtatgggatcaaactgctccgctgtttatgggtgagcacatgaaaaaaacactcattgcatgtactctgcaattcggaatatttgccacatctaacggcatgtacatgtggtttcccgatatcataagtaaaatgacagaatttcaaaacgctcatccaggagtaccaagtacaatatgctacgttgtccaaaattcatcaatgcttagggcggacgacttcattgatactacgaatagtacgaccgagtgtaaagacacaatggaagaacaggcatttatgcattctttaatgttggaagctggatatgcgattggatttcccataataggtgctattattaactctgtgggaaagcttccaattcttgtattcgtgatggtctcatgcgggatttgtggaataatttgtgcatttattgaacatttaacaatagcatcatatttatatttgtggttgctggtttgcggaattgctgtgacagtggtaaacgcggcattagtagatctgtaccctacacaactcagggcaatggcagtatgtatatctttaatgatgggtcgattaggaagtgttgttggaagcaatgttgtaggaataattctagattataattgcgatttaacatttttaatatcaggaacatctctgatagcatgtggagtgattgcattctttataccgaacatttatcagaagcaagtggatagaagaacgagcattgcttcatatggacca

SEQ ID NO: 6 - DNA, 1590 bp, Ctenocephalides felis
tggtccatatgaagcaatgctcgttcttctatccacttgcttctgataaatgttcggtataaagaatgcaatcactccacatgctatcagagatgttcctgatattaaaaatgttaaatcgcaattataatctagaattattcctacaacattgcttccaacaacacttcctaatcgacccatcattaaagatatacatactgccattgccctgagttgtgtagggtacagatctactaatgccgcgtttaccactgtcacagcaattccgcaaaccagcaaccacaaatataaatatgatgctattgttaaatgttcaataaatgcacaaattattccacaaatcccgcatgagaccatcacgaatacaagaattggaagctttcccacagagttaataatagcacctattatgggaaatccaatcgcatatccagcttccaacattaaagaatgcataaatgcctgttcttccattgtgtctttacactcggtcgtactattcgtagtatcaatgaagtcgtccgccctaagcattgatgaattttggacaacgtagcatattgtacttggtactcctggatgagcgttttgaaattctgtcattttacttatgatatcgggaaaccacatgtacatgccgttagatgtggcaaatattccgaattgcagagtacatgcaatgagtgtttttttcatgtgctcacccataaacagcggagcagtttgatcccatacagttcttaaaaatgatttctttgttttatttgtattttcaccatccactcctggatattcatctaagattgatgatacggggaattcactttctggtttaccagtattaattctatacattttggcaataatttgacgagcttcttcgtttcttccctgattcatcaaaaatttgggactttctggtaatttccacaaagcaaaacaacaaagcagtgatggcaaaccacatgcaaccatatacaacctccagggcttgtatgtatatcccaaaaagtcaatatagaatgaccatttctgatttataattaaccatgcaaccgtcggtaatgcaagacacgcaaatccgaagatgcttgacgctcccataatagccctgctgcgatgcctaggattatgaaattctcctaaatatgcatatattgttgcacttcctccagatacgaaaaatccattgaaatatcgcagcaaaataaacagccaaactgaatttacaaaactagatgccaaggtacaaaaaaatgcaagaagaagtgtaggcataatcactttccgcctccctctcgtatcggctaaaaatccccataaatgtgaactgcttataatacctataaaagctatagcgcttaatattcctttttgttttgttgttaattccagatcacattgtgctgaaggaacgacaaaactcattccaagagtttccatcaaaacacatactaatacgcatccactcacgaccagaagaaggtaattgaattttccaaaacctgtgagtgccaacgcatcttcaaactgccttggttttggttctttggaagctattatttgaatccccgtgttactgtccat

SEQ ID NO: 7 - DNA, 550 bp, Ctenocephalides felis
  misc_feature (476)..(476): n = unknown at position 476
ttttacatcaggagtgtgtgcagcctgcatgttctttgtttacaataaacgtcacaatttaatcgtgtctgcagtcttcagctctgttatatcttgtggaaatgcagcgttggactgtcttatcaccgaggtgtttccaacaaatttgagggctactggtgtggcaatatctatggtagctgctcgacttggaggcataattggcaatgtggtaattgcaactttattagacatgtattgtccagccccgacatttattgtagcactactattggctggaggtggtctgatgtgtctatttttgcctaacacgaccagggaaccactttcataagaaaatctcatcgtataacagtcgggcgatataataaatatatttattcttctgctccatcccaaaaacttgattttgaatttaaatacttataaaataatgagtcttttcttataaaaatgtatataaataattatcctanagtcgttcattaattanttcattaatgatgacttcgttattaattaataactantaccnancatcnanaaaaaa

SEQ ID NO: 8 - DNA, 3314 bp, Ctenocephalides felis
  CDS (302)..(2620)
gttggcgggccatcaagcgcttcagaattatcgtaattgtcgaataaaatatatactttataatacattcacatattgtatattaatagttttaattttaaatttattactaaatagttttaatttttaataagtggtgatacaagatttagataagagcccgatcgtcccggccggtgataacagcgcaacgcccagcagagacttagcatcgagcgcccgcgtcggttctcaacccctggccaaatcgaattcattccgaacgcaggagacagccactcaaaaccctattcggcataggatggtacagactgaccctgggcccaggcgtttgcttaacagtgacgttaaagacgaccaaccaccgtcaacgaatgatgaaataagcccatctgcgactgctccaagttcaaacgtgaatgaaagcaatggaaacctcaccgaatcacaacccttaagccaaaatacttcaacaaatactccattagaaacatcaaatacctcactagcagaaagcagcagtaccaagtcagctgcagacgttaataatagtaaacaaaacacggatactcaaagtaaagatggcctgagtcctttactaaaaagggatttgacggcgtcaacaaaattcctaaagaatattagtcagttagaattgaaaggtcaggatgatggtgaagataaaaatgagttgaaggaccagacggaggtcagtcaacatgacttgactagtcaaaaaacattgactgatccgacaacattggttactagagctaatgacaacagtagcaaaaaaagttcattaaagctcaaaaatggtggttccgttaattcagacaaaaggggtaaaaatgacccagaaaagggtcaaagcgatccgtccaagaaaattgcggcggattttgaaacagccatagatatgactggatatggaaaattccattacgccctgctggcggtttgtggttttgtatccacgagtgaagaaatggacgtaatctcaatgtcatttattctaccatcagcccaatgcgatttgaaattaaatacgcacactaagggttggttgaatagtatcatatttataggtatgatgtttggtgcctatctgtggggctccctagcagacagcgtaggacgcaaaagggtcctcatagcaatttcgttcatgaacgccctttgcatagtagccagttcattttcacaatcttacggactatttatgttattcagatttctaaacggagctgctttaggaggaagcggtcctgtaatttggtcatatttcgccgaatttcaaccaaaggcaaaaaggggctcaatgcttagttcaatggcagctttttggaccttgggtaatctttgtgtagcaggtttggcttgggccgttataccccaagccagtttaggatatcacggagtccatttcacatataactcatggcgcatatttttgtcattgtgtgctgttccatcattaattgtagccgtgctgctgttctttctgccggagtctccaaaatttctcttggccagaggcaaacaagaggaagccctggaaatattcagaggcattttccacacaaacactggaaagccgcgcgaccagtttccagtttgcgaattgatacccgacgagccgttgcccggccaaacgccgcagcagcgttcgctcacgaacttgttccaacataacgacgcccaagaaacgaataatcaaattaaatcaagcggagatttaattaaatcagcatcgaaagaaaatgttatcatgtatttggatgctaagaagcaggaaaacgttgaaatttcctacaactccgataaaatgaagatcggaaaaaataaaaggaaaggaaagtatagtcggatgttgtcagatatggctgagaacagcaagcagttgttcagaccgccaattttaagattcacattgatatcgataacgatcaacttcacgtttcacatcggttactatggtctgatgatgtggttcccagagctcttcaaccgtttcgacgaatacacacgccaccatttaactgacggaaacgaagtaacagccaatgtttgcgaagtaaccaactttgtagtttcacgtcatttggatcaacaccatgacgactttggagtcaccaagggggcctccggcccagtctgcgacgacgtcattcctgctgctgtgttcctggagtccctcataactgtcgcagcagccattcccagtaacataattgcagtcataggaatggatagacttggcagaaaattcttcttagttttcagtacttttacatcaggagtgtgtgcagcctgcatgttctttgtttacaataaacgtcacaatttaatcgtgtctgcagtcttcagctctgttatatcttgtggaaatgcagcgttggactgtcttatcaccgaggtgtttccaacaaatttgagggctactggtgtggcaatatctatggtagctgctcgacttggaggcataattggcaatgtggtaattgcaactttattagacatgtattgtccagccccgacatttattgtagcactactattggctggaggtggtctgatgtgtctatttttgcctaacacgaccagagaaccactttcataagaaaatctcatcgtataacagtcaggcgatataataaatatatttattcttctgctccatcccaaaaacttgattttgaaatttaaatacttataaaataatgagtcttttcttataaaaatgtatataaataattatccctaaagtcgttcaataattatttcattaatgatgacttcgttattaattaataactaaaaattaattaaacaaaaaatatacctatatgccacatctgtaaatgaaaacgaattataatataacataggatatcttaaaaaaagcactaaaattttttatatttataaaatttaaattaataaattccttcatattaattccattaaatcgtctaagtcttctcaatagtttagtatataatatgtgaatgtattataaatatgtattttattcataaggcaacaactaaaggcgtctttataatatacctaaaaaaatacaaaaatataaaaatacttgccaagtaatattttaataagtaataagttaagtgggaataaaaaatatttgtgttaatggtttaaaattttggttattcacataattttagaattgattcatgttgaatgtatgatgtaaaatttccctttaatttaagaataatagttctttatttgtttaatttcaatgtagttgatgaaataaattaaaaatgataaaaaaaaaaaaa

SEQ ID NO: 9 - protein, 773 aa, Ctenocephalides felis
MVQTDPGPRRLLNSDVKDDQPPSTNDEISPSATAPSSNVNESNGNLTESQPLSQNTSTNTPLETSNTSLAESSSTKSAADVNNSKQNTDTQSKDGLSPLLKRDLTASTKFLKNISQLELKGQDDGEDKNELKDQTEVSQHDLTSQKTLTDPTTLVTRANDNSSKKSSLKLKNGGSVNSDKRGKNDPEKGQSDPSKKIAADFETAIDMTGYGKFHYALLAVCGFVSTSEEMDVISMSFILPSAQCDLKLNTHTKGWLNSIIFIGMMFGAYLWGSLADSVGRKRVLIAISFMNALCIVASSFSQSYGLFMLFRFLNGAALGGSGPVIWSYFAEFQPKAKRGSMLSSMAAFWTLGNLCVAGLAWAVIPQASLGYHGVHFTYNSWRIFLSLCAVPSLIVAVLLFFLPESPKFLLARGKQEEALEIFRGIFHTNTGKPRDQFPVCELIPDEPLPGQTPQQRSLTNLFQHNDAQETNNQIKSSGDLIKSASKENVIMYLDAKKQENVEISYNSDKMKIGKNKRKGKYSRMLSDMAENSKQLFRPPILRFTLISITINFTFHIGYYGLMMWFPELFNRFDEYTRHHLTDGNEVTANVCEVTNFVVSRHLDQHHDDFGVTKGASGPVCDDVIPAAVFLESLITVAAAIPSNIIAVIGMDRLGRKFFLVFSTFTSGVCAACMFFVYNKRHNLIVSAVFSSVISCGNAALDCLITEVFPTNLRATGVAISMVAARLGGIIGNVVIATLLDMYCPAPTFIVALLLAGGGLMCLFLPNTTREPLS

SEQ ID NO: 10 - DNA, 3314 bp, Ctenocephalides felis
tttttttttttttatcatttttaatttatttcatcaactacattgaaattaaacaaataaagaactattattcttaaattaaagggaaattttacatcatacattcaacatgaatcaattctaaaattatgtgaataaccaaaattttaaaccattaacacaaatattttttattcccacttaacttattacttattaaaatattacttggcaagtatttttatatttttgtatttttttaggtatattataaagacgcctttagttgttgccttatgaataaaatacatatttataatacattcacatattatatactaaactattgagaagacttagacgatttaatggaattaatatgaaggaatttattaatttaaattttataaatataaaaaattttagtgctttttttaagatatcctatgttatattataattcgttttcatttacagatgtggcatataggtatattttttgtttaattaatttttagttattaattaataacgaagtcatcattaatgaaataattattgaacgactttagggataattatttatatacatttttataagaaaagactcattattttataagtatttaaatttcaaaatcaagtttttgggatggagcagaagaataaatatatttattatatcgcctgactgttatacgatgagattttcttatgaaagtggttctctggtcgtgttaggcaaaaatagacacatcagaccacctccagccaatagtagtgctacaataaatgtcggggctggacaatacatgtctaataaagttgcaattaccacattgccaattatgcctccaagtcgagcagctaccatagatattgccacaccagtagccctcaaatttgttggaaacacctcggtgataagacagtccaacgctgcatttccacaagatataacagagctgaagactgcagacacgattaaattgtgacgtttattgtaaacaaagaacatgcaggctgcacacactcctgatgtaaaagtactgaaaactaagaagaattttctgccaagtctatccattcctatgactgcaattatgttactgggaatggctgctgcgacagttatgagggactccaggaacacagcagcaggaatgacgtcgtcgcagactgggccggaggcccccttggtgactccaaagtcgtcatggtgttgatccaaatgacgtgaaactacaaagttggttacttcgcaaacattggctgttacttcgtttccgtcagttaaatggtggcgtgtgtattcgtcgaaacggttgaagagctctgggaaccacatcatcagaccatagtaaccgatgtgaaacgtgaagttgatcgttatcgatatcaatgtgaatcttaaaattggcggtctgaacaactgcttgctgttctcagccatatctgacaacatccgactatactttcctttccttttattttttccgatcttcattttatcggagttgtaggaaatttcaacgttttcctgcttcttagcatccaaatacatgataacattttctttcgatgctgatttaattaaatctccgcttgatttaatttgattattcgtttcttgggcgtcgttatgttggaacaagttcgtgagcgaacgctgctgcggcgtttggccgggcaacggctcgtcgggtatcaattcgcaaactggaaactggtcgcgcggctttccagtgtttgtgtggaaaatgcctctgaatatttccagggcttcctcttgtttgcctctggccaagagaaattttggagactccggcagaaagaacagcagcacggctacaattaatgatggaacagcacacaatgacaaaaatatgcgccatgagttatatgtgaaatggactccgtgatatcctaaactggcttggggtataacggcccaagccaaacctgctacacaaagattacccaaggtccaaaaagctgccattgaactaagcattgagcccctttttgcctttggttgaaattcggcgaaatatgaccaaattacaggaccgcttcctcctaaagcagctccgtttagaaatctgaataacataaatagtccgtaagattgtgaaaatgaactggctactatgcaaagggcgttcatgaacgaaattgctatgaggacccttttgcgtcctacgctgtctgctagggagccccacagataggcaccaaacatcatacctataaatatgatactattcaaccaacccttagtgtgcgtatttaatttcaaatcgcattgggctgatggtagaataaatgacattgagattacgtccatttcttcactcgtggatacaaaaccacaaaccgccagcagggcgtaatggaattttccatatccagtcatatctatggctgtttcaaaatccgccgcaattttcttggacggatcgctttgacccttttctgggtcatttttaccccttttgtctgaattaacggaaccaccatttttgagctttaatgaactttttttgctactgttgtcattagctctagtaaccaatgttgtcggatcagtcaatgttttttgactagtcaagtcatgttgactgacctccgtctggtccttcaactcatttttatcttcaccatcatcctgacctttcaattctaactgactaatattctttaggaattttgttgacgccgtcaaatccctttttagtaaaggactcaggccatctttactttgagtatccgtgttttgtttactattattaacgtctgcagctgacttggtactgctgctttctgctagtgaggtatttgatgtttctaatggagtatttgttgaagtattttggcttaagggttgtgattcggtgaggtttccattgctttcattcacgtttgaacttggagcagtcgcagatgggcttatttcatcattcgttgacggtggttggtcgtctttaacgtcactgttaagcaaacgcctgggcccagggtcagtctgtaccatcctatgccgaatagggttttgagtggctgtctcctgcgttcggaatgaattcgatttggccaggggttgagaaccgacgcgggcgctcgatgctaagtctctgctgggcgttgcgctgttatcaccggccgggacgatcgggctcttatctaaatcttgtatcaccacttattaaaaattaaaactatttagtaataaatttaaaattaaaactattaatatacaatatgtgaatgtattataaagtatatattttattcgacaattacgataattctgaagcgcttgatggcccgccaac

SEQ ID NO: 11 - DNA, 2319 bp, Ctenocephalides felis
atggtacagactgaccctgggcccaggcgtttgcttaacagtgacgttaaagacgaccaaccaccgtcaacgaatgatgaaataagcccatctgcgactgctccaagttcaaacgtgaatgaaagcaatggaaacctcaccgaatcacaacccttaagccaaaatacttcaacaaatactccattagaaacatcaaatacctcactagcagaaagcagcagtaccaagtcagctgcagacgttaataatagtaaacaaaacacggatactcaaagtaaagatggcctgagtcctttactaaaaagggatttgacggcgtcaacaaaattcctaaagaatattagtcagttagaattgaaaggtcaggatgatggtgaagataaaaatgagttgaaggaccagacggaggtcagtcaacatgacttgactagtcaaaaaacattgactgatccgacaacattggttactagagctaatgacaacagtagcaaaaaaagttcattaaagctcaaaaatggtggttccgttaattcagacaaaaggggtaaaaatgacccagaaaagggtcaaagcgatccgtccaagaaaattgcggcggattttgaaacagccatagatatgactggatatggaaaattccattacgccctgctggcggtttgtggttttgtatccacgagtgaagaaatggacgtaatctcaatgtcatttattctaccatcagcccaatgcgatttgaaattaaatacgcacactaagggttggttgaatagtatcatatttataggtatgatgtttggtgcctatctgtggggctccctagcagacagcgtaggacgcaaaagggtcctcatagcaatttcgttcatgaacgccctttgcatagtagccagttcattttcacaatcttacggactatttatgttattcagatttctaaacggagctgctttaggaggaagcggtcctgtaatttggtcatatttcgccgaatttcaaccaaaggcaaaaaggggctcaatgcttagttcaatggcagctttttggaccttgggtaatctttgtgtagcaggtttggcttgggccgttataccccaagccagtttaggatatcacggagtccatttcacatataactcatggcgcatatttttgtcattgtgtgctgttccatcattaattgtagccgtgctgctgttctttctgccggagtctccaaaatttctcttggccagaggcaaacaagaggaagccctggaaatattcagaggcattttccacacaaacactggaaagccgcgcgaccagtttccagtttgcgaattgatacccgacgagccgttgcccggccaaacgccgcagcagcgttcgctcacgaacttgttccaacataacgacgcccaagaaacgaataatcaaattaaatcaagcggagatttaattaaatcagcatcgaaagaaaatgttatcatgtatttggatgctaagaagcaggaaaacgttgaaatttcctacaactccgataaaatgaagatcggaaaaaataaaaggaaaggaaagtatagtcggatgttgtcagatatggctgagaacagcaagcagttgttcagaccgccaattttaagattcacattgatatcgataacgatcaacttcacgtttcacatcggttactatggtctgatgatgtggttcccagagctcttcaaccgtttcgacgaatacacacgccaccatttaactgacggaaacgaagtaacagccaatgtttgcgaagtaaccaactttgtagtttcacgtcatttggatcaacaccatgacgactttggagtcaccaagggggcctccggcccagtctgcgacgacgtcattcctgctgctgtgttcctggagtccctcataactgtcgcagcagccattcccagtaacataattgcagtcataggaatggatagacttggcagaaaattcttcttagttttcagtacttttacatcaggagtgtgtgcagcctgcatgttctttgtttacaataaacgtcacaatttaatcgtgtctgcagtcttcagctctgttatatcttgtggaaatgcagcgttggactgtcttatcaccgaggtgtttccaacaaatttgagggctactggtgtggcaatatctatggtagctgctcgacttggaggcataattggcaatgtggtaattgcaactttattagacatgtattgtccagccccgacatttattgtagcactactattggctggaggtggtctgatgtgtctatttttgcctaacacgaccagagaaccactttca

SEQ ID NO: 12 - DNA, 2319 bp, Ctenocephalides felis
tgaaagtggttctctggtcgtgttaggcaaaaatagacacatcagaccacctccagccaatagtagtgctacaataaatgtcggggctggacaatacatgtctaataaagttgcaattaccacattgccaattatgcctccaagtcgagcagctaccatagatattgccacaccagtagccctcaaatttgttggaaacacctcggtgataagacagtccaacgctgcatttccacaagatataacagagctgaagactgcagacacgattaaattgtgacgtttattgtaaacaaagaacatgcaggctgcacacactcctgatgtaaaagtactgaaaactaagaagaattttctgccaagtctatccattcctatgactgcaattatgttactgggaatggctgctgcgacagttatgagggactccaggaacacagcagcaggaatgacgtcgtcgcagactgggccggaggcccccttggtgactccaaagtcgtcatggtgttgatccaaatgacgtgaaactacaaagttggttacttcgcaaacattggctgttacttcgtttccgtcagttaaatggtggcgtgtgtattcgtcgaaacggttgaagagctctgggaaccacatcatcagaccatagtaaccgatgtgaaacgtgaagttgatcgttatcgatatcaatgtgaatcttaaaattggcggtctgaacaactgcttgctgttctcagccatatctgacaacatccgactatactttcctttccttttattttttccgatcttcattttatcggagttgtaggaaatttcaacgttttcctgcttcttagcatccaaatacatgataacattttctttcgatgctgatttaattaaatctccgcttgatttaatttgattattcgtttcttgggcgtcgttatgttggaacaagttcgtgagcgaacgctgctgcggcgtttggccgggcaacggctcgtcgggtatcaattcgcaaactggaaactggtcgcgcggctttccagtgtttgtgtggaaaatgcctctgaatatttccagggcttcctcttgtttgcctctggccaagagaaattttggagactccggcagaaagaacagcagcacggctacaattaatgatggaacagcacacaatgacaaaaatatgcgccatgagttatatgtgaaatggactccgtgatatcctaaactggcttggggtataacggcccaagccaaacctgctacacaaagattacccaaggtccaaaaagctgccattgaactaagcattgagcccctttttgcctttggttgaaattcggcgaaatatgaccaaattacaggaccgcttcctcctaaagcagctccgtttagaaatctgaataacataaatagtccgtaagattgtgaaaatgaactggctactatgcaaagggcgttcatgaacgaaattgctatgaggacccttttgcgtcctacgctgtctgctagggagccccacagataggcaccaaacatcatacctataaatatgatactattcaaccaacccttagtgtgcgtatttaatttcaaatcgcattgggctgatggtagaataaatgacattgagattacgtccatttcttcactcgtggatacaaaaccacaaaccgccagcagggcgtaatggaattttccatatccagtcatatctatggctgtttcaaaatccgccgcaattttcttggacggatcgctttgacccttttctgggtcatttttaccccttttgtctgaattaacggaaccaccatttttgagctttaatgaactttttttgctactgttgtcattagctctagtaaccaatgttgtcggatcagtcaatgttttttgactagtcaagtcatgttgactgacctccgtctggtccttcaactcatttttatcttcaccatcatcctgacctttcaattctaactgactaatattctttaggaattttgttgacgccgtcaaatccctttttagtaaaggactcaggccatctttactttgagtatccgtgttttgtttactattattaacgtctgcagctgacttggtactgctgctttctgctagtgaggtatttgatgtttctaatggagtatttgttgaagtattttggcttaagggttgtgattcggtgaggtttccattgctttcattcacgtttgaacttggagcagtcgcagatgggcttatttcatcattcgttgacggtggttggtcgtctttaacgtcactgttaagcaaacgcctgggcccagggtcagtctgtaccat